(19) United States
(12) Patent Application Publication
(10) Pub. No.: US 2024/0016804 A1
(43) Pub. Date: Jan. 18, 2024

(54) METHODS AND MATERIALS FOR ASSESSING AND TREATING HYPERTENSIVE DISORDERS

(71) Applicant: Mayo Foundation for Medical Education and Research, Rochester, MN (US)

(72) Inventors: Vesna D. Garovic, Rochester, MN (US); Tamar Tchkonia, Rochester, MN (US); James L. Kirkland, Rochester, MN (US)

(21) Appl. No.: 17/632,958

(22) PCT Filed: Aug. 14, 2020
(86) PCT No.: PCT/US2020/046299

§ 371 Date: Feb. 4, 2022

Related U.S. Application Data
(60) Provisional application No. 62/887,491, filed on Aug. 15, 2019.

(51) Int. Cl. A61K 31/506; A61P 9/12; G01N 33/68; C12Q 1/6883

(52) U.S. Cl. CPC A61K 31/506; A61P 9/12; G01N 33/689; C12Q 1/6883; G01N 2800/368; C12Q 2600/158

(57) ABSTRACT

This document relates to methods and materials for assessing and/or treating hypertensive disorders of pregnancy (e.g., preeclampsia) and/or one or more comorbidities associated with a hypertensive disorder of pregnancy. For example, methods and materials for determining if a female mammal has, or is likely to develop, a hypertensive disorder of pregnancy are provided. In addition, methods for using one or more senotherapeutic agents to treat a female mammal having, or at risk of developing, a hypertensive disorder of pregnancy (e.g., preeclampsia) are provided.

### CROSS-REFERENCE TO RELATED APPLICATIONS

This application claims the benefit of U.S. Patent Application Ser. No. 62/887,491, filed on Aug. 15, 2019. The disclosure of the prior application is considered part of (and is incorporated by reference in) the disclosure of this application.

### STATEMENT REGARDING FEDERAL FUNDING

This invention was made with government support under AG13925 and HL136348 awarded by the National Institutes of Health. The government has certain rights in the invention.

### BACKGROUND

### 1. Technical Field

This document relates to methods and materials for assessing and/or treating hypertensive disorders of pregnancy (e.g., preeclampsia) and/or one or more comorbidities associated with a hypertensive disorder of pregnancy. For example, methods and materials provided herein can be used to determine if a female mammal has, or is likely to develop, a hypertensive disorder of pregnancy. In some cases, one or more senotherapeutic agents can be administered to a female mammal having, or at risk of developing, a hypertensive disorder of pregnancy (e.g., preeclampsia) to treat the female mammal.

### 2. Background Information

Hypertensive disorders of pregnancy, including preeclampsia, remain one of the leading causes of maternal and fetal morbidity and mortality worldwide (Report of the National High Blood Pressure Education Program Working Group on High Blood Pressure in Pregnancy, Am. J. Obstet. Gynecol., 183(1):S1-522 (2000)). For example, preeclampsia (PE), a pregnancy-specific disorder characterized by hypertension and, commonly, proteinuria, commonly occurs after 20 weeks of gestation, and can affect approximately 5% of all pregnancies (Shohara et al., Cytotherapy, 14(10):1171-81 (2012)). Preeclampsia is frequently treated with blood pressure treatment, and premature delivery or termination of pregnancy is often recommended.

### SUMMARY

Preeclampsia can be difficult to diagnose, and there is no specific treatment of preeclampsia or its related health complications that occur later in life. This document provides methods and materials related to assessing and/or treating hypertensive disorders of pregnancy (e.g., preeclampsia) and/or one or more comorbidities associated with a hypertensive disorder of pregnancy. In some cases, this document provides methods and materials for identifying a female mammal as having, or as being likely to develop, a hypertensive disorder of pregnancy. For example, this document provides methods and materials for detecting the presence or absence of an elevated level of expression of one or more senescence-associated secretory phenotype (SASP) polypeptides within a female mammal (e.g., a female human) and classifying the female mammal as having, or as being likely to develop, a hypertensive disorder of pregnancy if the presence of an elevated level of expression of one or more SASP factor polypeptides is detected. In some cases, this document provides methods and materials for treating a female mammal having, or at risk of developing, a hypertensive disorder of pregnancy and/or one or more comorbidities associated with a hypertensive disorder of pregnancy. For example, this document provides methods and materials for using one or more senotherapeutic agents to treat a female mammal having, or at risk of developing, a hypertensive disorder of pregnancy and/or one or more comorbidities associated with a hypertensive disorder of pregnancy.

As described herein, the presence of an elevated level of expression of one or more SASP polypeptides in a sample obtained from a female mammal can be used to identify that female mammal as having, or as being at risk of developing, a hypertensive disorder of pregnancy and/or one or more comorbidities associated with a hypertensive disorder of pregnancy. For example, mesenchymal stem cells (MSCs) isolated from adipose tissue of women having preeclampsia can have a lower percentage of live MSCs as compared to MSCs isolated from adipose tissue of women having a normotensive pregnancy, and the live MSCs can exhibit a senescent phenotype, upregulation of senescence markers and SASP components, and lower angiogenic potential. As also described herein, one or more senotherapeutic agents such as dasatinib can be used to treat a hypertensive disorder of pregnancy. For example, treatment of MSCs isolated from adipose tissue of women having preeclampsia with dasatinib can increase the number of apoptotic MSCs and decreased expression of p16 and six SASP components as compared to MSCs isolated from adipose tissue of women having a normotensive pregnancy.

Having the ability to identify a female mammal as having or as being at risk of developing preeclampsia and/or having the ability to delay or prevent the development of a hypertensive disorder of pregnancy such as preeclampsia provides a unique and unrealized opportunity to allow women to carry a pregnancy to term safely, with fewer small-for-gestational age infants, and fewer stillbirths. In some cases, a non-pregnant woman at risk of developing a hypertensive disorder of pregnancy (e.g., preeclampsia) can be administered one or more senotherapeutic agents to prevent the development of such a disorder during pregnancy. For example, a non-pregnant woman who has had one or more previous unsuccessful pregnancies due to a hypertensive disorder of pregnancy (e.g., preeclampsia) can be administered one or more senotherapeutic agents prior to becoming pregnant to prevent the development of preeclampsia in a next pregnancy.

In general, one aspect of this document features methods for assessing a female mammal for a hypertensive disorder of pregnancy or the likelihood of developing the hypertensive disorder of pregnancy. The methods can include, or consist essentially of, (a) detecting the presence or absence of an elevated level of expression of a SASP polypeptide in a sample from the mammal; (b) classifying the mammal as having, or as likely to develop, the hypertensive disorder of pregnancy based at least in part on the presence of the elevated level, and (c) classifying the mammal as lacking, or as unlikely to develop, the hypertensive disorder of pregnancy based at least in part on the absence of the elevated level. The mammal can be a human. The mammal can be not pregnant. The mammal can be pregnant. The hypertensive disorder of pregnancy can be preeclampsia. The SASP polypeptide can be interleukin (IL)-6, IL-8, monocyte chemotactic protein-1 (MCP-1), plasminogen activator inhibitor-1 (PAI-1), osteopontin, activine A, eutaxin, GDF15, IL1 alpha, IL1 beta, MIPa, Rantese, MMP2 miRNA, MMP3 miRNA, MMP9 miRNA, or MMP12 miRNA. The sample can be whole blood, serum, plasma, peripheral blood mononucleated cells (PBMCs), urine, cerebrospinal fluid (CSF), adipose tissue, or skin tissue. In another aspect, this document features methods for treating a non-pregnant female mammal having a hypertensive disorder of pregnancy or likely to develop the hypertensive disorder of pregnancy. The methods can include, or consist essentially of, (a) detecting an elevated level of expression of a SASP polypeptide in a sample obtained from a mammal; and (b) administering a senolytic agent to the mammal. The mammal can be a human. The hypertensive disorder of pregnancy can be preeclampsia. The senolytic agent can be dasatinib. The SASP polypeptide can be IL-6, IL-8, MCP-1, PAI-1, osteopontin, activine A, eutaxin, GDF15, IL1 alpha, IL1 beta, MIPa, Rantese, MMP2 miRNA, MMP3 miRNA, MMP9 miRNA, or MMP12 miRNA. The sample can be whole blood, serum, plasma, PBMCs, urine, CSF, adipose tissue, or skin tissue.

In another aspect, this document features methods for treating a hypertensive disorder of pregnancy. The methods can include, or consist essentially of, administering a senolytic treatment to a non-pregnant female mammal identified as having an elevated level of expression of a SASP polypeptide in a sample obtained from the mammal. The mammal can be a human. The hypertensive disorder of pregnancy can be preeclampsia. The senolytic agent can be dasatinib. The SASP polypeptide can be IL-6, IL-8, MCP-1, PAI-1, osteopontin, activine A, eutaxin, GDF15, IL1 alpha, IL1 beta, MIPa, Rantese, MMP2 miRNA, MMP3 miRNA, MMP9 miRNA, or MMP12 miRNA. The sample can be whole blood, serum, plasma, PBMCs, urine, CSF, adipose tissue, or skin tissue.

In another aspect, this document features methods for delaying the onset of a comorbidity of a hypertensive disorder of pregnancy. The methods can include, or consist essentially of, administering a senolytic treatment to a non-pregnant female mammal (a) having had a pregnancy affected by a hypertensive disorder of pregnancy, and (b) identified as having an elevated level of expression of a SASP polypeptide in a sample obtained from the mammal. The mammal can be a human. The hypertensive disorder of pregnancy can be preeclampsia. The senolytic agent can be dasatinib. The comorbidity of the hypertensive disorder of pregnancy can be diabetes mellitus type 2, cardiovascular disease, stroke, kidney disease, vascular dysfunction, atherosclerotic burden, fat inflammation, vascular injury, cardiac arrhythmias, congestive heart failure, dementia, depression, substance abuse, hyperlipidemia, arthritis, cancer, asthma, chronic obstructive pulmonary disease, or osteoporosis. The SASP polypeptide can be IL-6, IL-8, MCP-1, PAI-1, osteopontin, activine A, eutaxin, GDF15, IL1 alpha, IL1 beta, MIPa, Rantese, MMP2 miRNA, MMP3 miRNA, MMP9 miRNA, or MMP12 miRNA. The sample can be whole blood, serum, plasma, PBMCs, urine, CSF, adipose tissue, or skin tissue.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Difference in biological aging (epigenetic age at delivery−epigenetic age at first trimester) during pregnancy in normotensive and preeclamptic women. BA=biological age.

FIG. 2. Staining for senescence-associated ß-galactosidase (SABG) in MSCs from women with normotensive pregnancies (NP) vs. women with PE with before and after treatment with senolytics. Scale bar=200 μm.

FIG. 3. Angiogenesis in MSCs from women with NP vs. women with PE before and after treatment with senolytics.

FIG. 4. Inclusion criteria and analysis cohorts. A pregnancy was classified as having sufficient information to determine HDP status if there was at least one blood pressure measurement from a prenatal visit and at least one blood pressure measurement from admission for delivery.

FIG. 5. Age-specific per-pregnancy incidence of hypertensive disorders of pregnancy among 9,862 pregnancies during 1976-1982 for residents of Olmsted County, Minnesota. HTN=hypertension.

FIG. 6. Cumulative incidence curves for cardiovascular and metabolic conditions in women with hypertensive disorders of pregnancy compared with 1:2 age- and parity-matched referent women. The reported hazard ratios (HR) and corresponding 95% CIs were estimated from Cox models adjusted for age, education, smoking, and obesity.

FIG. 7. Accumulation of multimorbidity (unadjusted Aalen-Johansen curves). The accumulation of chronic conditions is shown as the mean number of conditions accumulated over the follow-up after the index date considering all 16 chronic conditions (left-hand graph) and considering all 16 chronic conditions except HTN (right-hand graph).

FIG. 8. Expression of inflammatory cytokines in non-pregnant woman MSC treated for 24 hours with TNF-alpha. All 3 markers tested were significantly elevated after treatment with TNF-alpha vs. vehicle (presented as mean±SD): IL-6, 11.73±2.20 vs. 1.22±0.47 (p=0.009); IL-8: 6.29±2.53 vs. 0.36±0.47 (p=0.38); MCP-1: 38.07±7.46 vs. 1.65±1.46 (p=0.010), respectively.

FIGS. 9A-9B. Fat tissue staining for markers of inflammation and oxidative stress in NP (upper rows) and PE women (lower rows). TNF-alpha and MCP-1 were up-regulated in PE. Representative images for TNF-alpha, MCP-1, DAPI (4,6-Diamidino-2-phenylindole, dihydrochloride) nuclear stain (nucleus) staining, as well as and merged TNF-alpha and MCP-1 (FIG. 9A). DHE (Dihydroethidium) staining tended to be increased in PE compared to NP. Representative images for DHE and DAPI, as well as merged DHE and DAPI (FIG. 9B).

FIG. 10. Representative flow cytometry scatterplots of MSC viability. MSC viability tested using Annexin V (channel 11) and Sytox (Channel 2) show decreased MSC viability in preeclamptic (85%) vs. normotensive (94%) pregnancies (p=0.01). This is a representative image where live cells, dead cells and apoptotic cells are as labeled.

FIGS. 11A-11D. Senescent cell burden is higher in PE-MSC compared to NP-MSC. SABG staining showed a higher number of stained cells (marked with black arrows) in PE-MSC compared to normotensive counterparts (FIG. 11A). Data presented as mean values of SABG-stained MSC with min-max (FIG. 11B). Expression of p16, but not p21, was significantly increased. All SASP genes were significantly more expressed in PE-MSC compared to NP-MSC. Data shown as box-plots (min-max) with all individual values (FIG. 11C). PE-MSC and NP-MSC were co-cultured with GFP expressing HUVEC for 8 days in total and total network length was measured continuously every 3 hours. Significantly lower angiogenic potential was registered for HUVEC co-cultured with PE-MSC, compared to NP-MSC (F=13.965; df=8, p<0.001) (FIG. 11D).

FIGS. 12A-12B. Apoptotic effects of the senolytic agent, Dasatinib, on MSCs. Dose response experiments showed that PE-MSCs are sensitive to a lower concentration (1 μM) of Dasatinib, while the apoptotic effect of the drug is lower at higher concentrations of the drug (FIG. 12A). Treatment with luM Dasatinib revealed significant apoptotic effects in PE-MSCs (p=0.0117) compared to the non-treated cells, but not in NP-MSCs (p=0.0934). Representative image shows apoptotic cells stained red for PE-MSCs and NP-MSCs in all three conditions (medium, vehicle, treatment) (n=3) (FIG. 12B).

FIGS. 13A-13C. Angiogenic potential of Dasatinib-treated PE-MSCs was improved after the treatment. Representative images showing the total network length formed at day 0 and day 8 after senolytic agent treatment of PE-MSCs and NP-MSCs (FIG. 13A). While no significant difference in angiogenic potential of NP-MSCs (n=9) was observed after the treatment (F=0.406; df=8;p=0.916) (FIG. 13B), NP-MSCs co-cultured HUVEC showed significant improvement in angiogenesis (F=22.436; df=8; p<0.001) (FIG. 13C).

FIGS. 14A-14D. Treatment with Dasatinib cleared senescent cells from PE-MSCs and affected senescence related gene expression. Representative images from SA-β-gal staining show abundant senescent cells in PE-MSCs (marked with black arrows), but not in NP-MSCs, in vehicle (FIG. 14A). Dasatinib treatment completely removed senescent cells from PE-MSCs (p<0.001) (FIG. 14B). PE-MSCs had significant decreases in expression of p16 (p=0.025) and PAI-1 (p<0.001), IL-6 (p=0. 0487), and MCP-1 (p=0.040), while IL-8 (p=0.136) was decreased after treatment. Significantly increased expression of the p21 and PAI2 genes was observed after the treatment (FIG. 14C). Relative expression of the senescence marker gene, p16, in NP-MSCs after treatment with Dasatinib remained unchanged (p=0.136). Apart from p21 and PAI2 whose expression increased in a manner similar to PE-MSCs, the relative gene expressions of the other tested genes was decreased in NP-MSCs after the treatment with Dasatinib (p<0.001) (FIG. 14D).

FIGS. 15A-15B. Representative images of abdominal fat tissue biopsies stained for p16. Red arrows indicate positive (brown) staining for p16 in fat tissues sections in preeclamptic, but not in normotensive pregnancies. FIG. 15A. Normotensive Patient. FIG. 15B. Preeclampsia Patient.

FIG. 16. Luminex 100 analysis of SASP components indicates upregulation of both MCP-1 and TNF-alpha after protein isolation from the fat tissue samples from preeclamptic (n=10) compared to normotensive (n=12) pregnancies.

### DETAILED DESCRIPTION

This document provides methods and materials for identifying and/or treating female mammals (e.g., female humans) having, or at risk of developing, a hypertensive disorder of pregnancy (e.g., preeclampsia) and/or one or more comorbidities associated with a hypertensive disorder of pregnancy. In some cases, this document provides methods and materials for identifying a female mammal as having, or at risk of developing, a hypertensive disorder of pregnancy and/or one or more comorbidities associated with a hypertensive disorder of pregnancy. As described herein, an elevated level of expression of one or more SASP polypeptides can be present in a sample obtained from a female mammal having, or at risk of developing, a hypertensive disorder of pregnancy and/or one or more comorbidities associated with a hypertensive disorder of pregnancy. In some cases, this document provides methods and materials for identifying a female mammal as having, or at risk of developing, a hypertensive disorder of pregnancy and/or one or more comorbidities associated with a hypertensive disorder of pregnancy. For example, a female mammal (e.g., a female human) can be identified as having, or at risk of developing, a hypertensive disorder of pregnancy and/or one or more comorbidities associated with a hypertensive disorder of pregnancy based, at least in part, on the presence of an elevated level of expression of one or more SASP polypeptides in a sample obtained from the female mammal. In some cases, this document provides methods and materials for treating a female mammal (e.g., a female human) having, or at risk of developing, a hypertensive disorder of pregnancy and/or one or more comorbidities associated with a hypertensive disorder of pregnancy. For example, a female mammal identified as having, or at risk of developing, a hypertensive disorder of pregnancy and/or one or more comorbidities associated with a hypertensive disorder of pregnancy as described herein (e.g., based, at least in part, on the presence of an elevated level of expression of one or more SASP polypeptides in a sample from the female mammal) can be administered one or more senotherapeutic agents (e.g., dasatinib) to treat the female mammal. In some cases, one or more senotherapeutic agents can be used to treat a female mammal having had a previous pregnancy affected by a hypertensive disorder of pregnancy (e.g., prior to a subsequent pregnancy to prevent or delay developing a hypertensive disorder of pregnancy in that subsequent pregnancy).

In some cases, the presence of an elevated level of expression of one or more (e.g., one, two, three, four, or more) SASP polypeptides in a sample (e.g., a sample obtained from a female mammal such as a female human) can be used to identify the female mammal as having, or as being likely to develop, a hypertensive disorder of pregnancy and/or one or more comorbidities associated with a hypertensive disorder of pregnancy. The term “elevated level” as used herein with respect to a level of expression of one or more SASP polypeptides refers to any level that is greater than a reference level of expression of a SASP polypeptide. The term “reference level” as used herein with respect to expression of a SASP polypeptide refers to the level of expression of the SASP polypeptide typically observed in a sample (e.g., a control sample) from one or more comparable female mammals (e.g., female humans of comparable age) that do not have a to hypertensive disorder of pregnancy and/or one or more comorbidities associated with a hypertensive disorder of pregnancy. Control samples can include, without limitation, samples from female mammals having a normotensive pregnancy, and samples from non-pregnant female mammals. In some cases, an elevated level of expression of a SASP polypeptide can be a level that is at least 2 (e.g., at least 5, at least 10, at least 15, at least 20, at least 25, at least 35, or at least 50) fold greater than a reference level of expression of the SASP polypeptide. In some cases, an elevated level of expression of a SASP polypeptide can be a level that is greater than 1 picogram (pg) per μL. In some cases, when control samples have an undetectable level of expression of a SASP polypeptide, an elevated level can be any detectable level of expression of the SASP polypeptide. It will be appreciated that levels from comparable samples are used when determining whether or not a particular level is an elevated level. In some cases, an elevated level of expression of a SASP polypeptide for a sample can be a level where greater than 0.0001 percent of the cells of the sample express detectable levels of expression of SASP polypeptides. For example, when greater than 0.0001 percent of the cells of a sample are determined to express a detectable level of expression of a SASP polypeptide, then that sample can be classified as having an elevated level of expression of the SASP polypeptide.

The presence of an elevated level of expression of any appropriate SASP polypeptide (e.g., in a sample such as a sample obtained from a female mammal such as a female human) can be used to identify a female mammal as having, or as being likely to develop, a hypertensive disorder of pregnancy (e.g., preeclampsia) and/or one or more comorbidities associated with a hypertensive disorder of pregnancy. Examples of SASP polypeptides include, without limitation, IL-6, IL-8, MCP-1, PAI-1, osteopontin, activine A, eutaxin, GDF15, IL1 alpha, IL1 beta, MIPa, Rantese, MMP2 miRNA, MMP3 miRNA, MMP9 miRNA, and MMP12 miRNA. Exemplary polypeptide sequences (and the nucleic acids encoding such polypeptides) of SASP polypeptides can be as set forth in the National Center for Biotechnology Information (NCBI) databases at, for example, Accession No. NM 001318095.1, Accession No. NM_002982, and Accession No. NM_001165413.

Any appropriate mammal can be assessed and/or treated as described herein. In some cases, a female mammal can be a pregnant mammal. For example, when a senotherapeutic agent is senomorphic agent, a female mammal can be a pregnant mammal. In some cases, a female mammal can be a non-pregnant mammal. For example, when a senotherapeutic agent is senolytic agent, a female mammal can be a non-pregnant mammal. In some cases, a female mammal can have experienced one or more previous pregnancies (e.g., one or more previous pregnancies affected by a hypertensive disorder of pregnancy). In some cases, a female mammal can be an obese female mammal (e.g., a female mammal that is overweight). Examples of female mammals that can be treated using a composition containing one or more senotherapeutic agents as described herein include, without limitation, humans, non-human primates such as monkeys, dogs, cats, horses, cows, pigs, sheep, mice, and rats. In some cases, a composition containing one or more senotherapeutic agents can be administered to a human having a hypertensive disorder of pregnancy (e.g., preeclampsia) to treat the human. In some cases, a composition containing one or more senotherapeutic agents can be administered to a female human at risk of developing one or more comorbidities associated with a hypertensive disorder of pregnancy to slow the onset or progression of the one or more comorbidities associated with a hypertensive disorder of pregnancy within the human.

Any appropriate sample from a female mammal (e.g., a female human) can be assessed as described herein (e.g., for the presence, absence, or level of expression of one or more SASP polypeptides). In some cases, a sample can be a biological sample. In some cases, a sample can contain one or more biological molecules (e.g., nucleic acids such as DNA and RNA, polypeptides, carbohydrates, lipids, hormones, and/or metabolites). Examples of samples that can be assessed as described herein include, without limitation, fluid samples (e.g., whole blood, serum, plasma, PBMCs, urine, and CSF), and tissue samples (e.g., adipose tissue, and skin tissue). A biological sample can be a fresh sample or a fixed sample (e.g., a formaldehyde-fixed sample or a formalin-fixed sample). In some cases, a biological sample can be a processed sample (e.g., an embedded sample such as a paraffin or OCT embedded sample, or processed to isolate or extract one or more biological molecules). For example, a blood (e.g., serum) sample can be obtained from a female mammal and can be assessed for the presence, absence, or level of expression of one or more SASP polypeptides to determine if the mammal has, or is likely to develop, a hypertensive disorder of pregnancy such as preeclampsia based, at least in part, on the presence of an elevated level of expression of one or more SASP polypeptides in the sample.

Any appropriate method can be used to detect the presence, absence, or level of expression of one or more SASP polypeptides within a sample (e.g., a sample obtained from a female mammal such as a female human). In some cases, the presence, absence, or level of expression of one or more SASP polypeptides within a sample can be determined by detecting the presence, absence, or level of one or more SASP polypeptides in the sample. For example, immunoassays (e.g., immunohistochemistry (IHC) techniques, and western blotting techniques), mass spectrometry techniques (e.g., proteomics-based mass spectrometry assays or targeted quantification-based mass spectrometry assays), and enzyme-linked immunosorbent assays (ELISAs) can be used to determine the presence, absence, or level of one or more SASP polypeptides in a sample. When an immunoassay is used to determine the presence, absence, or level of one or more SASP polypeptides in a sample, the immunoassay can use any appropriate antibody. Examples of antibodies that can be used in an immunoassay to determine the presence, absence, or level of one or more SASP polypeptides in a sample include, without limitation, human activin A antibodies (e.g., R&D systems DAC008), human total MMP-7 antibodies (e.g., R&D systems DMP700), human IL-6 antibodies (e.g., R&D systems

D6050), human serpin E1/PAI-1 antibodies (e.g., R&D systems DSE100), human CCL2/MCP-1 antibodies (e.g., R&D systems DCP00), human osteopontin antibodies (e.g., eBioscience BMS2066), and human MMP-1 antibodies (e.g., RayBiotech., Inc. ELH-MMP-1).

In some cases, the presence, absence, or level of expression of one or more SASP polypeptides within a sample can be determined by detecting the presence, absence, or level of mRNA encoding a SASP polypeptide in the sample. For example, polymerase chain reaction (PCR)-based techniques such as quantitative reverse transcription (RT)-PCR (qPCR) techniques, RNA in situ hybridization (ISH), and RNA sequencing can be used to determine the presence, absence, or level of mRNA encoding a SASP polypeptide in the sample. In some cases, the presence, absence, or level of expression of one or more SASP polypeptides within a sample can be determined by qPCR. In some cases, the presence, absence, or level of expression of one or more SASP polypeptides within a sample can be determined as described in Example 3.

In some cases, when a female mammal (e.g., a female human) is identified as having, or as being likely to develop, a hypertensive disorder of pregnancy (e.g., preeclampsia) and/or one or more comorbidities associated with a hypertensive disorder of pregnancy based, at least in part, on an elevated level of expression of one or more SASP polypeptides in a sample as described herein, the presence of a hypertensive disorder of pregnancy and/or one or more comorbidities associated with a hypertensive disorder of pregnancy can be confirmed using one or more diagnostic techniques. Any appropriate technique can be used to confirm that a female mammal has, or is at risk of developing, a hypertensive disorder of pregnancy (e.g., preeclampsia) and/or one or more comorbidities associated with a hypertensive disorder of pregnancy. For example, proteinuria, a low platelet count, impaired liver function, signs of kidney problems (e.g., other than proteinuria), pulmonary edema, new-onset headaches or visual disturbances, a blood pressure in excess of 140/90 mm Hg and/or seizures after the 20th week of pregnancy can be used to confirm a female mammal as having a hypertensive disorder of pregnancy. For example, a history of preeclampsia (e.g., during a previous pregnancy), chronic hypertension, first pregnancy, new paternity (e.g., as compared to a previous pregnancy), age (e.g., pregnancy in women younger than 20 and/or pregnancy in women older than 40), obesity, pregnancy with multiples (e.g., twins, triplets, or other multiples), interval between pregnancies (e.g., pregnancies less than two years or more than 10 years apart), history of certain medical conditions (e.g., chronic high blood pressure, migraines, type 1 or type 2 diabetes, and/or kidney disease) prior to pregnancy, and/or conception by in vitro fertilization can be used to confirm a female mammal as being at risk of developing a hypertensive disorder of pregnancy. For example, a female mammal having had a hypertensive disorder of pregnancy (e.g., preeclampsia) during one or more previous pregnancies can be confirmed as being at risk of developing one or more comorbidities associated with a hypertensive disorder of pregnancy.

When assessing and/or treating a hypertensive disorder of pregnancy as described herein, the hypertensive disorder of pregnancy can be any type of hypertensive disorder of pregnancy. Examples of hypertensive disorders of pregnancy that can be treated as described herein include, without limitation, preeclampsia, postpartum preeclampsia, eclampsia, chronic hypertension, preeclampsia superimposed on chronic hypertension, and gestational hypertension (e.g., transient hypertension of pregnancy or chronic hypertension identified in the latter half of pregnancy).

When assessing and/or treating a comorbidity associated with a hypertensive disorder of pregnancy as described herein, the comorbidity associated with a hypertensive disorder of pregnancy can be any type of comorbidity associated with a hypertensive disorder of pregnancy. In some cases, a comorbidity can be an age-related disease. In some cases, a comorbidity associated with a hypertensive disorder of pregnancy can be present at the same time as a hypertensive disorder of pregnancy (e.g., during the pregnancy). In some cases, a comorbidity associated with a hypertensive disorder of pregnancy can develop after the pregnancy. Examples of comorbidities (e.g., future comorbidities such as one or more comorbidities that can develop following the completion of the female mammal's pregnancy) associated with a hypertensive disorder of pregnancy that can be treated as described herein include, without limitation, hypertension, diabetes (e.g., diabetes mellitus type 2), vascular disease (e.g., cardiovascular disease (CVD) such as coronary artery disease), stroke, kidney disease (e.g., chronic kidney disease), vascular dysfunction, atherosclerotic burden, inflammation (e.g., fat inflammation and/or systemic inflammation), vascular injury, cardiac arrhythmias, congestive heart failure, dementia, and hyperlipidemia.

Once identified as having, or as being at risk of developing, a hypertensive disorder of pregnancy (e.g., preeclampsia) and/or one or more comorbidities associated with a hypertensive disorder of pregnancy, a female mammal can be administered or instructed to self-administer one or more (e.g., one, two, three, four, five or more) senotherapeutic agents. Any appropriate senotherapeutic agent can be used as described herein. A senotherapeutic agent that can be used as described herein can be any type of molecule (e.g., small molecules or polypeptides). In some cases, a senotherapeutic agent can be a senolytic agent (i.e., an agent having the ability to induce cell death in senescent cells). In some cases, a senotherapeutic agent can be a senomorphic agent (i.e., an agent having the ability to suppress senescent phenotypes without cell killing). Examples of senotherapeutic agents that can be used as described herein (e.g., to treat a female mammal having, or at risk of developing, a hypertensive disorder of pregnancy such as preeclampsia and/or one or more comorbidities associated with a hypertensive disorder of pregnancy) can include, without limitation, dasatinib, quercetin, navitoclax, A1331852, A1155463, fisetin, luteolin, geldanamycin, tanespimycin, alvespimycin, piperlongumine, panobinostat, FOX04-related peptides, nutlin3a, ruxolitinib, metformin, and rapamycin.

One or more senotherapeutic agents (e.g., dasatinib) can be administered to a female mammal having, or at risk of developing, a hypertensive disorder of pregnancy (e.g., preeclampsia) and/or one or more comorbidities associated with a hypertensive disorder of pregnancy at any appropriate time. In some cases, one or more senotherapeutic agents can be administered to a female mammal (e.g., a female human) in need thereof (e.g., a female mammal having, or at risk of developing, a hypertensive disorder of pregnancy) as described herein prior to the female mammal becoming pregnant. In some cases, one or more senotherapeutic agents can be administered to a female mammal (e.g., a female human) in need thereof (e.g., a female mammal having, or at risk of developing, a hypertensive disorder of pregnancy) as described herein during the female mammal's pregnancy. In some cases, one or more senotherapeutic agents can be administered to a female mammal (e.g., a female human) in need thereof (e.g., a female mammal having, or at risk of developing, a hypertensive disorder of pregnancy) as described herein following the completion of the female mammal's pregnancy.

In some cases, a female mammal having, or at risk of developing, a hypertensive disorder of pregnancy can be treated with one or more senotherapeutic agents (e.g., dasatinib) to alleviate (e.g., to reduce or eliminate) one or more (e.g., one, two, three, four, five or more) symptoms of the hypertensive disorder of pregnancy. A symptom of a hypertensive disorder of pregnancy can be any appropriate symptom. Examples of symptoms of preeclampsia include, without limitation, hypertension, proteinuria, headaches, blurred vision, temporary loss of vision, sensitivity to light, abdominal pain, nausea and/or vomiting, decreased urine output, thrombocytopenia, impaired liver function, shortness of breath (e.g., caused by fluid in the lungs), and edema (e.g., pulmonary edema). Each of these symptoms of a hypertensive disorder of pregnancy can be identified and/or monitored using clinical techniques as described elsewhere (see, e.g., Am. J. Obstet. Gynecol., 183(1):S1-S22 (2000)) For example, one or more senotherapeutic agents can be administered to a female mammal (e.g., a female human) in need thereof (e.g., a female mammal having, or at risk of developing, a hypertensive disorder of pregnancy) as described herein to reduce the severity of one or more symptoms a hypertensive disorder of pregnancy in the female mammal by, for example, 10, 20, 30, 40, 50, 60, 70, 80, 90, 95, or more percent.

In some cases, a female mammal having, or at risk of developing, a comorbidity associated with a hypertensive disorder of pregnancy (e.g., a female mammal having had a pregnancy affected by a hypertensive disorder of pregnancy) can be treated with one or more senotherapeutic agents (e.g., dasatinib) to delay or prevent the onset of a comorbidity associated with a hypertensive disorder of pregnancy. For example, a female mammal (e.g., a female human) who has previously had one or more previous pregnancies affected by a hypertensive disorder of pregnancy can be administered one or more senotherapeutic agents to delay the onset of a comorbidity associated with a hypertensive disorder of pregnancy by about 7 to about 10 years (e.g., as compared to the age of development of the same comorbidity in a female mammal who experienced a comparable hypertensive disorder of pregnancy and was not administered the one or more senotherapeutic agents). For example, a female mammal (e.g., a female human) who has previously had one or more previous pregnancies affected by a hypertensive disorder of pregnancy can be administered one or more senotherapeutic agents to prevent the onset of a comorbidity associated with a hypertensive disorder of pregnancy (e.g., as compared to the age of development of the same comorbidity in a female mammal who experienced a comparable hypertensive disorder of pregnancy and was not administered the one or more senotherapeutic agents).

In some cases, a female mammal having, or at risk of developing, a comorbidity associated with a hypertensive disorder of pregnancy (e.g., a female mammal having had a pregnancy affected by a hypertensive disorder of pregnancy) can be treated with one or more senotherapeutic agents (e.g., dasatinib) to alleviate (e.g., to reduce or eliminate) one or more (e.g., one, two, three, four, five or more) symptoms of the comorbidity associated with a hypertensive disorder of pregnancy. A symptom of a comorbidity associated with a hypertensive disorder of pregnancy can be any appropriate symptom. Examples of symptoms of hypertension associated with a hypertensive disorder of pregnancy include, without limitation, headaches, chest pain, shortness of breath or nosebleeds, epigastric pain, dizziness, and palpitations. Each of these symptoms of hypertension associated with a hypertensive disorder of pregnancy can be identified and/or monitored using clinical techniques as described elsewhere (see, e.g., Am. J. Obstet. Gynecol., 183(1):S1-S22 (2000)). Examples of symptoms of diabetes (e.g., diabetes mellitus type 2) associated with a hypertensive disorder of pregnancy include, without limitation, increased thirst, frequent urination, increased hunger, unintended weight loss, fatigue, blurred vision, slow-healing sores, frequent infections, areas of darkened skin, and fainting spells (e.g., due to hypoglycemic episodes). Each of these symptoms of diabetes (e.g., diabetes mellitus type 2) associated with a hypertensive disorder of pregnancy can be identified and/or monitored using clinical techniques as described elsewhere (see, e.g., Weissgerber et al., Curr Diab Rep. 15(3):9 (2015)). Examples of symptoms of a CVD such as coronary artery disease associated with a hypertensive disorder of pregnancy include, without limitation, chest pain (angina), shortness of breath, heart attack, pulmonary edema, and cardiac arrhythmias. Each of these symptoms of coronary artery disease associated with a hypertensive disorder of pregnancy can be identified and/or monitored using clinical techniques as described elsewhere (see, e.g., Mosca et al., Circulation. 99(18):2480-4 (1999)). Examples of symptoms of stroke associated with a hypertensive disorder of pregnancy include, without limitation, trouble with speaking and understanding, paralysis or numbness (e.g., of the face, arm or leg), trouble with seeing in one or both eyes, headache, trouble with walking, trouble with swallowing, confusion, and loss of conciseness. Each of these symptoms of stroke associated with a hypertensive disorder of pregnancy can be identified and/or monitored using clinical techniques as described elsewhere (see, e.g., Bushnell et al., Stroke. 45(5):1545-88. (2014)). Examples of symptoms of kidney disease associated with a hypertensive disorder of pregnancy include, without limitation, nausea, vomiting, loss of appetite, fatigue and weakness, sleep problems, changes in amounts of urinate, decreased mental sharpness, muscle twitches and cramps, swelling of feet and ankles, persistent itching, chest pain (e.g., if fluid builds up around the lining of the heart), shortness of breath (e.g., if fluid builds up in the lungs), metallic taste, decreased appetite, and weight loss. Each of these symptoms of kidney disease associated with a hypertensive disorder of pregnancy can be identified and/or monitored using clinical techniques as described elsewhere (see, e.g., Kidney Disease: Improving Global Outcomes (KDIGO) CKD-MBD Work, Kidney Int Suppl, August 2009 (113):S1-S130 (2009)). For example, one or more senotherapeutic agents can be administered to a female mammal (e.g., a female human) in need thereof (e.g., a female mammal having, or at risk of developing, a comorbidity associated with a hypertensive disorder of pregnancy) as described herein to reduce the severity of one or more symptoms of a comorbidity associated with a hypertensive disorder of pregnancy in the female mammal by, for example, 10, 20, 30, 40, 50, 60, 70, 80, 90, 95, or more percent.

In some cases, a female mammal having, or at risk of developing, a hypertensive disorder of pregnancy (e.g., preeclampsia) and/or one or more comorbidities associated with a hypertensive disorder of pregnancy can be treated with one or more senotherapeutic agents (e.g., dasatinib) to clear one or more senescent cells from within the female mammal. A senescent cell can be any type of cell. Examples of senescent cells that can be cleared as described herein include, without limitation, preadipocytes, endothelial cells, dendritic cells, fibroblasts, astrocytes, myofibroblast, pancreatic beta cells, and osteoblasts. A senescent cell can be cleared from any location within the female mammal. Examples of locations from which a senescent cell can be cleared include, without limitation, adipose tissue, vascular system, bone, marrow, brain, heart, pancreas, liver, kidney, muscle, and blood of the female mammal. In some cases, administering one or more senotherapeutic agents to a female mammal having, or at risk of developing, a hypertensive disorder of pregnancy and/or one or more comorbidities associated with a hypertensive disorder of pregnancy can be effective to clear one or more senescent cells from adipose tissue within the female mammal. For example, one or more senotherapeutic agents can be administered to a female mammal (e.g., a female human) in need thereof (e.g., a female mammal having, or at risk of developing, a hypertensive disorder of pregnancy and/or a comorbidity associated with a hypertensive disorder of pregnancy) as described herein to clear, for example, 10, 20, 30, 40, 50, 60, 70, 80, 90, 95, or more percent of the senescent cells present from a location (e.g., adipose tissue) within the female mammal.

In some cases, a female mammal having, or at risk of developing, a hypertensive disorder of pregnancy (e.g., preeclampsia) and/or one or more comorbidities associated with a hypertensive disorder of pregnancy can be treated with one or more senotherapeutic agents (e.g., dasatinib) to increase (e.g., restore) angiogenesis (e.g., to increase the angiogenic potential of a cell) within the female mammal. In some cases, a female mammal having, or at risk of developing, a hypertensive disorder of pregnancy (e.g., preeclampsia) and/or one or more comorbidities associated with a hypertensive disorder of pregnancy can be treated with a composition including one or more senotherapeutic agents (e.g., dasatinib) to alleviate (e.g., to reduce or eliminate) age-related impairment of angiogenesis in the female mammal. Angiogenesis can be increased in any appropriate type of cell. In some cases, angiogenesis can be increased in a stem cell. Examples of cells in which angiogenesis can be increased as described herein include, without limitation, MSCs (e.g., an adipose-derived MSC), and endothelial cells. For example, clearance of one or more senescent cells from a population of cells including MSCs and/or endothelial cells can increase angiogenesis of the MSCs and/or the endothelial cells. In some cases, administering one or more senotherapeutic agents to a female mammal having, or at risk of developing, a hypertensive disorder of pregnancy (e.g., preeclampsia) and/or one or more comorbidities associated with a hypertensive to disorder of pregnancy can be effective to increase angiogenesis in one or more MSCs within the female mammal. For example, one or more senotherapeutic agents can be administered to a female mammal (e.g., a female human) in need thereof (e.g., a female mammal having, or at risk of developing, a hypertensive disorder of pregnancy and/or a comorbidity associated with a hypertensive disorder of pregnancy) as described herein to increase angiogenesis in, for example, 10, 20, 30, 40, 50, 60, 70, 80, 90, 95, or more percent of cells (e.g., MSCs) present within the female mammal.

In some cases, a female mammal having, or at risk of developing, a hypertensive disorder of pregnancy (e.g., preeclampsia) and/or one or more comorbidities associated with a hypertensive disorder of pregnancy can be treated with one or more senotherapeutic agents (e.g., dasatinib) to alleviate (e.g., to reduce or eliminate) inflammation in the female mammal. A level (e.g., a systemic level) of any appropriate inflammatory factor (e.g., cytokines, chemokines, and matrix proteases) can be altered (e.g., increased or decreased) to alleviate inflammation in a female mammal having, or at risk of developing, a hypertensive disorder of pregnancy and/or one or more comorbidities associated with a hypertensive disorder of pregnancy. In cases where an inflammatory factor is a pro-inflammatory factor (e.g., SASP polypeptides such as IL-6, IL-8, MCP-1, PAI-1, GDF15, MIPa, eutaxin, osteopontin, and activine A), the pro-inflammatory factor can be decreased. In cases where an inflammatory factor is an anti-inflammatory factor, the anti-inflammatory factor can be increased. For example, one or more senotherapeutic agents can be administered to a female mammal (e.g., a female human) in need thereof (e.g., a female mammal having, or at risk of developing, a hypertensive disorder of pregnancy and/or a comorbidity associated with a hypertensive disorder of pregnancy) as described herein to alter a level of an inflammatory factor in a cell within the female mammal by, for example, 10, 20, 30, 40, 50, 60, 70, 80, 90, 95, or more percent. Inflammation at any appropriate location within the female mammal can be alleviated. Examples of locations from which inflammation can be alleviated as described herein include, without limitation, adipose tissue, brain, pancreas, liver, kidney, vasculature, blood, brain, and CSF. In some cases, administering one or more senotherapeutic agents to a female mammal having, or at risk of developing, a hypertensive disorder of pregnancy and/or one or more comorbidities associated with a hypertensive disorder of pregnancy can be effective to alleviate adipose inflammation within the female mammal.

In some cases, one or more senotherapeutic agents (e.g., dasatinib) can be formulated into a composition (e.g., a pharmaceutically acceptable composition) for administration to a female mammal having, or at risk of developing, a hypertensive disorder of pregnancy (e.g., preeclampsia) and/or one or more comorbidities associated with a hypertensive disorder of pregnancy. For example, one or more senotherapeutic agents can be formulated together with one or more pharmaceutically acceptable carriers (additives), excipients, and/or diluents. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil.

A composition including one or more senotherapeutic agents (e.g., dasatinib) can be designed for any type of administration to a female mammal having, or at risk of developing, a hypertensive disorder of pregnancy (e.g., preeclampsia) and/or one or more comorbidities associated with a hypertensive disorder of pregnancy. For example, a composition including one or more senotherapeutic agents can be designed for oral or parenteral (including, without limitation, a subcutaneous, intramuscular, intravenous, intradermal, intra-cerebral, intrathecal, or intraperitoneal (i.p.) injection) administration to a female mammal having, or at risk of developing, a hypertensive disorder of pregnancy and/or one or more comorbidities associated with a hypertensive disorder of pregnancy. Compositions suitable for oral administration include, without limitation, liquids, tablets, capsules, pills, powders, gels, and granules. In some cases, compositions suitable for oral administration can be in the form of a food supplement. In some cases, compositions suitable for oral administration can be in the form of a drink supplement. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

A composition including one or more senotherapeutic agents (e.g., dasatinib) can be administered to a female mammal having, or at risk of developing, a hypertensive disorder of pregnancy (e.g., preeclampsia) and/or one or more comorbidities associated with a hypertensive disorder of pregnancy in any appropriate amount (e.g., any appropriate dose). Effective amounts can vary depending on the route of administration, the age and general health condition of the subject, excipient usage, the possibility of co-usage with other therapeutic treatments such as use of other agents, and the judgment of the treating physician. An effective amount of a composition containing one or more senotherapeutic agents can be any amount that can treat a female mammal having, or at risk of developing, a hypertensive disorder of pregnancy and/or one or more comorbidities associated with a hypertensive disorder of pregnancy as described herein without producing significant toxicity to the female mammal. For example, an effective amount of dasatinib can be from about 1 micromolar (μM) to about 20 μM (e.g., from about 1 μM to about 15 μM, from about 1 μM to about 12 μM, from about 1 μM to about 10 μM, from about 1 μM to about 7 μM, from about 1 μM to about 5 μM, from about 5 μM to about 20 μM, from about 8 μM to about 20 μM, from about 10 μM to about 20 μM, from about 12 μM to about 20 μM, from about 15 μM to about 20 μM, from about 5 μM to about 15 μM, from about 8 μM to about 12 μM, from about 5 μM to about 10 μM, or from about 10 μM to about 15 μM). For example, an effective amount of dasatinib can be from about 1 milligrams per kilogram body weight (mg/kg) to about 20 mg/kg (e.g., about 5 mg/kg). The effective amount can remain constant or can be adjusted as a sliding scale or variable dose depending on the female mammal's response to treatment. Various factors can influence the actual effective amount used for a particular application. For example, the frequency of administration, duration of treatment, use of multiple treatment agents, route of administration, and/or severity of the hypertensive disorder of pregnancy (e.g., preeclampsia) and/or the one or more comorbidities associated with a hypertensive disorder of pregnancy in the female mammal being treated may require an increase or decrease in the actual effective amount administered.

A composition containing one or more senotherapeutic agents (e.g., dasatinib) can be administered to a female mammal having, or at risk of developing, a hypertensive disorder of pregnancy (e.g., preeclampsia) and/or one or more comorbidities associated with a hypertensive disorder of pregnancy in any appropriate frequency. The frequency of administration can be any frequency that can treat a female mammal having, or at risk of developing, a hypertensive disorder of pregnancy and/or one or more comorbidities associated with a hypertensive disorder of pregnancy without producing significant toxicity to the female mammal. For example, the frequency of administration can be from about once a day to about once a week, from about once a week to about once a month, or from about twice a month to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

A composition containing one or more senotherapeutic agents (e.g., dasatinib) can be administered to a female mammal having, or at risk of developing, a hypertensive disorder of pregnancy (e.g., preeclampsia) and/or one or more comorbidities associated with a hypertensive disorder of pregnancy for any appropriate duration. An effective duration for administering or using a composition containing one or more senotherapeutic agents can be any duration that can treat a female mammal having, or at risk of developing, a hypertensive disorder of pregnancy and/or one or more comorbidities associated with a hypertensive disorder of pregnancy without producing significant toxicity to the female mammal. For example, the effective duration can vary from several weeks to several months, from several months to several years, or from several years to a lifetime. In some cases, the effective duration can range in duration from about 10 years to about a lifetime. Multiple factors can influence the actual effective duration used for a particular treatment. For example, an effective duration can vary with the frequency of administration, effective amount, use of multiple treatment agents, and/or route of administration.

In some cases, a composition containing one or more (e.g., one, two, three, four, five or more) senotherapeutic agents (e.g., dasatinib) can include the one or more senotherapeutic agent(s) as the sole active ingredient(s) in the composition effective to treat a hypertensive disorder of pregnancy (e.g., preeclampsia) and/or one or more comorbidities associated with a hypertensive disorder of pregnancy.

In some cases, a composition containing one or more (e.g., one, two, three, four, five or more) senotherapeutic agents (e.g., dasatinib) can include one or more (e.g., one, two, three, four, five or more) additional active agents (e.g., therapeutic agents) in the composition that are effective to treat a hypertensive disorder of pregnancy (e.g., preeclampsia) and/or one or more comorbidities associated with a hypertensive disorder of pregnancy.

In some cases, a female mammal having, or at risk of developing, a hypertensive disorder of pregnancy (e.g., preeclampsia) and/or one or more comorbidities associated with a hypertensive disorder of pregnancy being treated as described herein (e.g., by administering one or more senotherapeutic agents such as dasatinib) also can be treated with one or more (e.g., one, two, three, four, five or more) additional therapeutic agents. A therapeutic agent used in combination with one or more senotherapeutic agents described herein can be any appropriate therapeutic agent. Examples of therapeutic agents that can be used in combination with one or more senotherapeutic agents described herein include, without limitation, antihypertensives, corticosteroids, anticonvulsant medications, aspirin (e.g., low-dose aspirin), calcium supplements, and magnesium sulfate. In some cases, the one or more additional therapeutic agents can be administered together with the one or more senotherapeutic agents (e.g., in a composition containing one or more senotherapeutic agents and containing one or more additional therapeutic agents). In some cases, the one or more (e.g., one, two, three, four, five or more) additional therapeutic agents can be administered independent of the one or more senotherapeutic agents. When the one or more additional therapeutic agents are administered independent of the one or more senotherapeutic agents, the one or more senotherapeutic agents can be administered first, and the one or more additional therapeutic agents administered second, or vice versa.

In some cases, methods for treating a female mammal (e.g., a female human) having, or at risk of developing, a hypertensive disorder of pregnancy (e.g., preeclampsia) and/or one or more comorbidities associated with a hypertensive disorder of pregnancy as described herein (e.g., by administering one or more senotherapeutic agents such as dasatinib) also can include subjecting the mammal to one or more (e.g., one, two, three, four, five or more) additional treatments (e.g., therapeutic interventions) that are effective to treat a hypertensive disorder of pregnancy (e.g., preeclampsia) and/or one or more comorbidities associated with a hypertensive disorder of pregnancy to treat the mammal. An example of an additional treatment that can be used as described herein to treat a hypertensive disorder of pregnancy (e.g., preeclampsia) and/or one or more comorbidities associated with a hypertensive disorder of pregnancy include, without limitation, stem cell (e.g., MSC) transplantation (e.g., autologous stem cell transplantation). In some cases, MSCs present in an autologous stem cell transplantation can be treated with one or more senotherapeutic agents. For example, when a female mammal identified as having, or at risk of developing, a hypertensive disorder of pregnancy and/or one or more comorbidities associated with a hypertensive disorder of pregnancy is a pregnant female mammal, the pregnant female mammal can be treated by providing the pregnant female mammal with an autologous MSC transplantation where the MSCs have been treated with one or more senotherapeutic agents. In some cases, the one or more additional treatments that are effective to treat one or more symptoms of a hypertensive disorder of pregnancy and/or one or more comorbidities associated with a hypertensive disorder of pregnancy can be performed at the same time as the administration of the one or more senotherapeutic agents. In some cases, the one or more additional treatments that are effective to treat one or more symptoms of a hypertensive disorder of pregnancy and/or one or more comorbidities associated with a hypertensive disorder of pregnancy can be performed before and/or after the administration of the one or more senotherapeutic agents.

In some cases, methods for treating a female mammal (e.g., a female human) having, or at risk of developing, hypertensive disorder of pregnancy (e.g., preeclampsia) and/or one or more comorbidities associated with a hypertensive disorder of pregnancy as described herein (e.g., by administering one or more senotherapeutic agents) also can include monitoring the female mammal being treated. Any appropriate method can be used to monitor the severity of a hypertensive disorder of pregnancy and/or one or more comorbidities associated with a hypertensive disorder of pregnancy in a female mammal. In some cases, methods described herein also can include monitoring a female mammal being treated as described herein for toxicity. The level of toxicity, if any, can be determined by assessing a female mammal's clinical signs and symptoms before and after administering a known amount of a particular composition. It is noted that the effective amount of a particular composition administered to a female mammal can be adjusted according to a desired outcome as well as the female mammal's response and level of toxicity.

The invention will be further described in the following examples, which do not limit the scope of the invention described in the claims.

### EXAMPLES

### Example 1: The Therapeutic Potential of Senolytics for Prevention of Preeclampsia and Treatment of Related Comorbidities in Post-Reproductive Age

This Example demonstrates that accelerated aging, in general, and cellular senescence, a cellular program characterized by irreversible proliferation arrest in particular, play a mechanistic role in the pathophysiology of preeclampsia (PE).

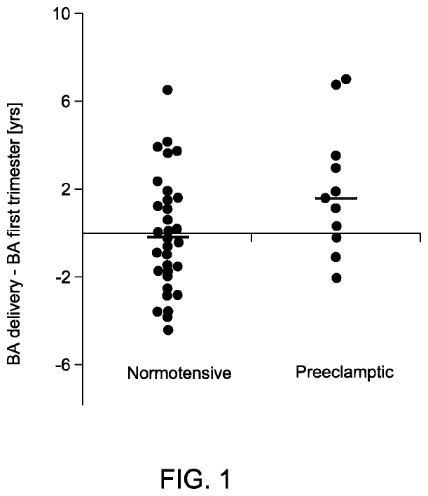

Mean epigenetic age (using a validated “epigenetic clock” method) was 2.4 years higher in women with preeclampsia (PE) vs. normotensive pregnancy (NP) at delivery (FIG. 1).

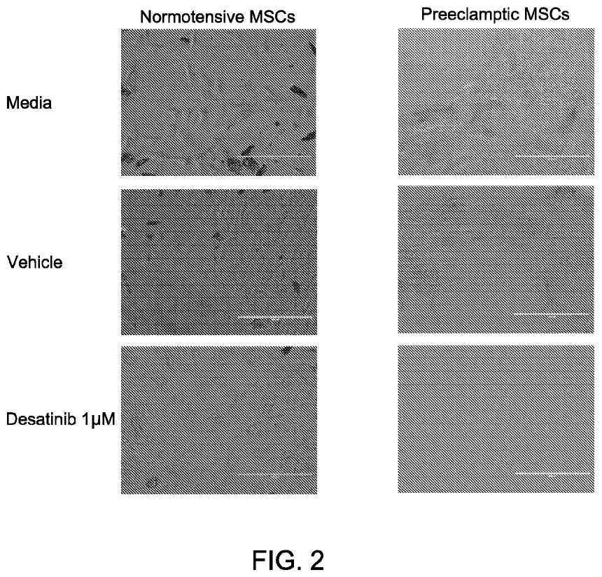

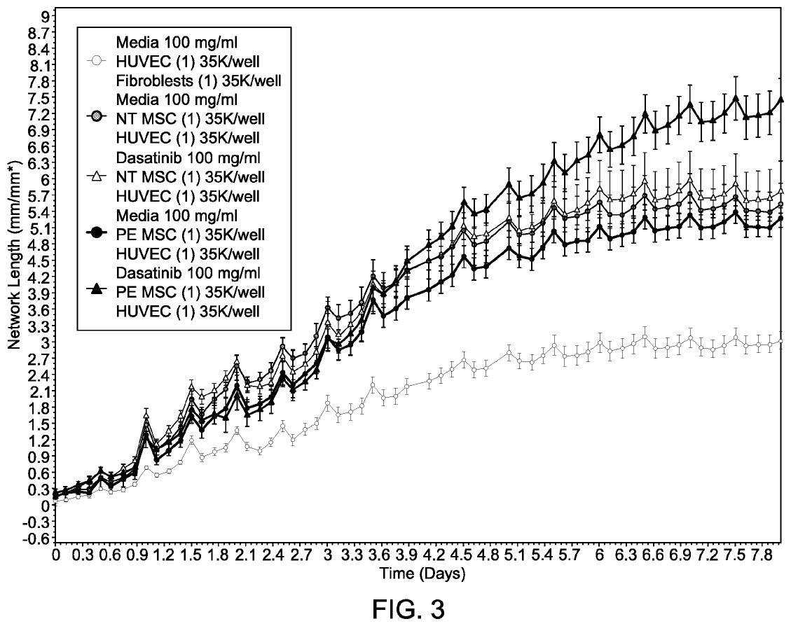

Cellular senescence and a senescence-associated secretory phenotype (SASP), a distinctive secretome consisting of inflammatory cytokines and reactive oxygen species that ultimately leads to vascular injury were also examined. MSCs, multipotent cells with immunomodulatory, anti-inflammatory, and pro-angiogenic activities, which were isolated from the abdominal fat tissue (3-5 g) removed during C-sections from normotensive and PE pregnancies were studied. An abundance of cells stained positively for senescence-associated B-galactosidase (SABG) cultured MSC from PE, but not in cultured MSC from NP. Treatment with senolytics significantly reduced the senescence cell number in PE (FIG. 2) and significantly improved their angiogenic potential as tested using tube formation assay (FIG. 3).

These results demonstrate that senolytics can be used as a targeted treatment for PE.

### Example 2: Hypertensive Disorders of Pregnancy: Incidence Per-Pregnancy and Per-Woman, Future Outcomes and Multimorbidity—A Population-Based Cohort Study

### Methods

### Study Design and Population

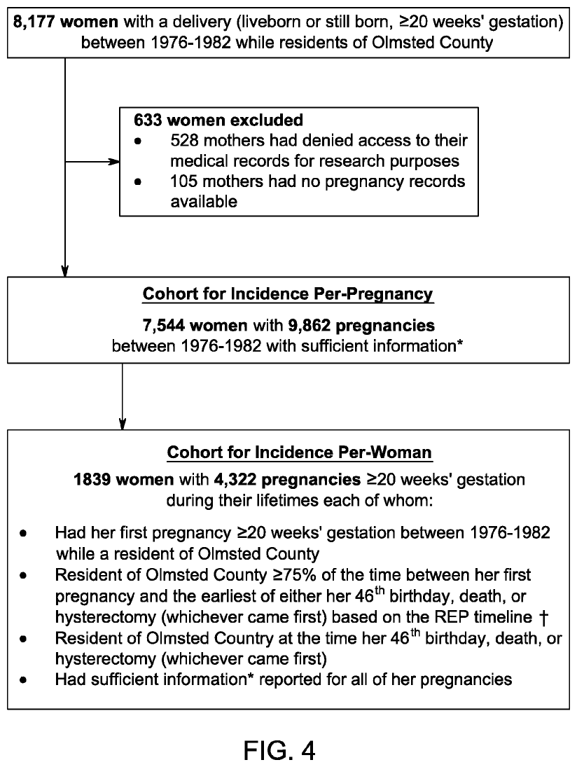

The Rochester Epidemiology Project (REP) medical record-linkage system was used to establish a cohort consisting of all women (n=8,177) who were residents of Olmsted County, Minnesota and delivered (liveborn or stillborn, ≥20 weeks' gestation) between Jan. 1, 1976 and Dec. 31, 1982 (FIG. 4). The record-linkage system of the REP has been described comprehensively elsewhere (St. Sauver et al., Am. J. Epidemiol. 173:1059-68 (2011); and St Sauver et al., Int. J. Epidemiol. 41:1614-24 (2012)). Briefly, the REP links all of the medical records from all providers in Olmsted County (encompassing Mayo Clinic and Olmsted Medical Center and their affiliated hospitals and medical facilities) using a unit medical record system whereby all outpatient, inpatient, emergency room, and nursing home information is kept in the same unit record. The 1976-1982 time period was selected for HDP assessment so that there was sufficient time for the women i) to complete their reproductive years, which would allow for the study of HDP incidence per woman and ii) to develop age-related chronic conditions in order to examine the relationship between HDP and adverse future outcomes. This study was approved by the Institutional Review Boards at Mayo Clinic and Olmsted Medical Center.

### Identification of Per-Pregnancy Cohort and Per-Woman Sub-Cohort for HDP Incidence

Women who either did not consent to the use of their medical records for research or with insufficient pregnancy information reported in the medical record were excluded. A pregnancy was classified as having sufficient information to determine HDP status if there was at least one blood pressure measurement from a prenatal visit and at least one blood pressure measurement from admission for delivery. The final cohort used for estimating the per-pregnancy incidence of HDP consisted of 7,544 mothers with 9,862 pregnancies. For the incidence of HDP per-woman, a sub-cohort of 1,839 women was identified. These women had their first deliveries between1976 and 1982 while residents of Olmsted County and who were also residents of Olmsted County by the end of their childbearing years, and for whom sufficient information was reported for all of their pregnancies.

Women with diagnostic codes indicative of a possible HDP occurring between Jan. 1, 1976 and Dec. 31, 1982, were first identified and their charts were fully abstracted (a list of codes is shown in Table 1). Every chart of each remaining woman in the cohort without a code suggestive of a possible HDP was then screened. A positive screen was defined as two elevated blood pressures, either systolic blood pressures (SBP)>140 mmHg and/or diastolic blood pressures (DBP)>90 mmHg at any prenatal visit, during admission for delivery, or postnatally before leaving the hospital. All screen positive charts were then fully abstracted. Screen-negative charts were categorized as normotensive pregnancies.

TABLE 1
Hospital International Classification of Diseases Adapted
(HICDA) codes utilized to identify potential cases of
HPD in the study population between 1976 and 1982.

HICDA Codes used
0650-0664 Full-term delivery of livebirth or stillbirth
and premature delivery of livebirth
or stillbirth after 20 weeks of gestation
4000-4050 Hypertension, malignant, accelerated
06370-06379 Preeclampsia, eclampsia, HELLP, toxemia
06330-06339 Intrauterine death
06343-06344 Intrauterine growth retardation
06352 Infarction, placental
06353 Insufficiency, placental (mother's history)
07600-07603 Premature infant, preterm
07640 Fetal distress before labor
07649 Birth asphyxia, unspecified
07747 High blood pressure, labile blood pressure
07756 Edema, swelling fluid retention
07700 Coma
07703 Convulsion, seizure
07733 Hyperreflexia, abnormal reflexes
Berkson codes used
013502 Dead, fetus
013512 Maceration, fetus
013554 Still birth

### Assignment of HDP Exposure Status

From the first prenatal visit through 12 weeks post-partum, all data regarding blood pressures, dipstick protein, and hypertensive medication use at each visit were recorded and dated. The following laboratory values were collected between the first prenatal visit and up to 72 hours post-partum: 24-hour protein, serum creatinine, platelet count, and liver function tests. A validated electronic diagnostic algorithm (Milic et al., Mayo Clin. Proc. 93:1707-19 (2018)) was used to determine the presence of any HDP and type based on accepted clinical criteria and the diagnosis of hypertension, which to required blood pressure elevations in greater than 50% of blood pressure readings (the “50% rule”). The algorithm was validated by comparison of algorithm-based diagnoses to the gold standard, i.e., physician-made diagnoses. The algorithm-based approach demonstrated significant improvements in sensitivity and specificity in the classification of exposure (i.e., HDP) compared to methods that utilized diagnostic codes only. The definition of each HDP type used in the algorithm is described in Table 2. In addition to the data that were required to classify pregnancies as normotensive vs. HDP, demographic, prenatal and intrapartum data were also collected. All abstracted data were first recorded on paper forms and subsequently entered into a database for future analysis.

TABLE 2
Definitions and diagnostic criteria of hypertensive disorders of pregnancy (see,
e.g., Hypertension in pregnancy, Obstet. Gynecol. 122: 1122-31 (2013)).

Chronic hypertension. Diagnosed based on data obtained at <20 weeks of gestation,
chronic hypertension was diagnosed if any of these three criteria were met during non-
Emergency room visits: (1) SBP >140 mm Hg or DBP >90 mm Hg on at least two
occasions within one month, but at least 6 hours apart; (2) prescription of an anti-
hypertensive medication or; (3) evidence of chronic hypertension prior to pregnancy
AND one SBP >140 mm Hg or DBP >90 mm Hg prior to 20 gestational weeks. Women
with chronic hypertension were eligible for the superimposed diagnoses of eclampsia or
preeclampsia.
Gestational hypertension. Diagnosed based on data obtained at >20 weeks of gestation,
gestational hypertension was diagnosed if any of these three criteria were met during non-
Emergency room visits: (1) sustained SBP >140 mm Hg or DBP >90 mm Hg in a
previously normotensive woman (occurred more than 50% of consecutive blood pressure
readings from the first recorded elevated prenatal blood pressure through 24 hours
postpartum); or (2) one SBP >140 mm Hg and/or DBP >90 mm Hg in a patient who was
also prescribed an anti-hypertensive medication for blood pressure control within one
week of the blood pressure elevation; or (3) by expert physician review if the patient had
one or more blood pressure elevations more than 48 hours after delivery, but was coded
as normotensive by the algorithm. The latter definition was included in an effort to
identify postpartum HDP.
Preeclampsia. Diagnosed based on data obtained at >20 weeks of gestation, preeclampsia
was defined as gestational hypertension plus (1) new or worsening proteinuria; (2)
administration of magnesium sulfate for seizure prophylaxis; or (3) new abnormal
laboratory testing (AST/ALT >70 U/L, platelet count <100 × 10(9)/L, or serum
creatinine >1.1 mg/dL). If the only evidence, in addition to gestational hypertension, was
subjective symptoms of persistent headache, epigastric pain or hyperreflexia, then a
diagnosis of possible preeclampsia was assigned. Preeclampsia superimposed on chronic
hypertension required worsening of BP control plus new onset or worsening proteinuria,
new abnormal lab tests, or subjective symptoms.
Eclampsia. Defined as preeclampsia plus new onset seizures

### Inter- and Intra-rater Reliability

There were six abstractors involved in retrieving data from the medical records over the course of the project. Both inter-rater and intra-rater reliability were assessed for the diagnosis of HDP and type. For example, there were 21 pregnancies abstracted by the same nurse on different days, several months apart. The diagnosis of HDP and type were the same for all 21 pregnancies, resulting in 100% intra-rater agreement. Additionally, 128 pregnancies were accessed by two different abstractors, of which 126 (98.4%) had the same diagnosis of HDP and type.

### Identification of Cohort for Outcome Analysis

For each woman with a pregnancy complicated by HDP, the index date was defined as the date when she first met criteria for HPD. Two referent women with normotensive pregnancies were randomly identified from the women in the birth cohort matched for the date of delivery (±1 year), maternal age (±1 year), and parity at index pregnancy (1 or >1) who had not met criteria for HPD before the index date. Women who first met criteria for HPD based on a delivery prior to 1976 were excluded from this analysis. Women with more than one normotensive pregnancy during 1976-1982 could have been identified as a matched referent for more than one HDP woman.

### Ascertainment of Chronic Conditions

Chronic conditions recommended by the US Department of Health and Human Services (DHHS) were considered for long-term multimorbidity (Table 3). The following 4 DHHS conditions were excluded because they were rare in the population: human immunodeficiency virus infections, autism spectrum disorders, schizophrenia, and hepatitis. These sixteen conditions were ascertained electronically by retrieving diagnosis codes from inpatient and outpatient visits to REP-affiliated providers from the index date through the women's last visits. To reduce the risk of false-positive diagnoses, a woman was classified as having a condition if she had at least 2 occurrences of a diagnosis code (either the same code or 2 different codes within the code set for a given condition) separated by more than 30 days, as described elsewhere (Rocca et al., Mayo Clin. Proc. 91:1577-89 (2016)). In addition to examining the relationship between HDP and each US DHHS condition, risk of multimorbidity (a measure of accelerated aging) and death were also examined. To capture those conditions that caused acute deaths, a single diagnosis code listed anywhere on a death certificate was also sufficient.

TABLE 3
Chronic conditions recommended by the US Department of Health and Human Services to
study multimorbidity (Weissgerber et al., Mayo Clin. Proc. 90: 1207-16 (2015)).

| Description * | CCC† | CMS (ICD-9 codes) ‡ |
|---|---|---|
| Hyperlipidemia | 53 | 272.0, 272.1, 272.2, 272.3, 272.4 |
| Hypertension | 98, 99 | 401.0, 401.1, 401.9, 402.00, 402.01, 402.10, 402.11, 402.90, |
| | | 402.91, 403.00, 403.01, 403.10, 403.11, 403.90, 403.91, |
| | | 404.00, 404.01, 404.02, 404.03, 404.10, 404.11, 404.12, |
| | | 404.13, 404.90, 404.91, 404.92, 404.93, 405.01, 405.09, |
| | | 405.11, 405.19, 405.91, 405.99, 362.11, 437.2 |
| Depression | 657 | 296.20, 296.21, 296.22, 296.23, 296.24, 296.25, 296.26, |
| | | 296.30, 296.31, 296.32, 296.33, 296.34, 296.35, 296.36, |
| | | 296.51, 296.52, 296.53, 296.54, 296.55, 296.56, 296.60, |
| | | 296.61, 296.62, 296.63, 296.64, 296.65, 296.66, 296.89, |
| | | 298.0, 300.4, 309.1, 311 |
| Diabetes | 49, 50 | 249.00, 249.01, 249.10, 249.11, 249.20, 249.21, 249.30, |
| | | 249.31, 249.40, 249.41, 249.50, 249.51, 249.60, 249.61, |
| | | 249.70, 249.71, 249.80, 249.81, 249.90, 249.91, 250.00, |
| | | 250.01, 250.02, 250.03, 250.10, 250.11, 250.12, 250.13, |
| | | 250.20, 250.21, 250.22, 250.23, 250.30, 250.31, 250.32, |
| | | 250.33, 250.40, 250.41, 250.42, 250.43, 250.50, 250.51, |
| | | 250.52, 250.53, 250.60, 250.61, 250.62, 250.63, 250.70, |
| | | 250.71, 250.72, 250.73, 250.80, 250.81, 250.82, 250.83, |
| | | 250.90, 250.91, 250.92, 250.93, 357.2, 362.01, 362.02, |
| | | 362.03, 362.04, 362.05, 362.06, 366.41 |
| Arthritis | 202, 203 | 714.0, 714.1, 714.2, 714.30, 714.31, 714.32, 714.33, 715.00, |
| | | 715.04, 715.09, 715.10, 715.11, 715.12, 715.13, 715.14, |
| | | 715.15, 715.16, 715.17, 715.18, 715.20, 715.21, 715.22, |
| | | 715.23, 715.24, 715.25, 715.26, 715.27, 715.28, 715.30, |
| | | 715.31, 715.32, 715.33, 715.34, 715.35, 715.36, 715.37, |
| | | 715.38, 715.80, 715.89, 715.90, 715.91, 715.92, 715.93, |
| | | 715.94, 715.95, 715.96, 715.97, 715.98, 720.0, 721.0, 721.1, |
| | | 721.2, 721.3, 721.90, 721.91 |
| Cancer | 11-43 | Female breast cancer: 174.0, 174.1, 174.2, 174.3, 174.4, |
| | | 174.5, 174.6, 174.8, 174.9, 175.0, 175.9, 233.0, V10.3. |
| | | Colorectal cancer: 154.0, 154.1, 153.0, 153.1, 153.2, 153.3, |
| | | 153.4, 153.5, 153.6, 153.7, 153.8, 153.9, 230.3, 230.4, V10.05. |
| | | Prostate cancer: 185, 233.4, V10.46. |
| | | Lung cancer:162.2, 162.3, 162.4, 162.5, 162.8, 162.9, 231.2, |
| | | V10.11. |
| Cardiac | 105, 106 | 427.31 |
| arrhythmia | | |
| Asthma | 128 | 493.00, 493.01, 493.02, 493.10, 493.11, 493.12, 493.20, |
| | | 493.21, 493.22, 493.81, 493.82, 493.90, 493.91, 493.92 |
| Coronary | 100, 101 | 410.00, 410.01, 410.02, 410.10, 410.11, 410.12, 410.20, |
| artery disease | | 410.21, 410.22, 410.30, 410.31, 410.32, 410.40, 410.41, |
| | | 410.42, 410.50, 410.51, 410.52, 410.60, 410.61, 410.62, |
| | | 410.70, 410.71, 410.72, 410.80, 410.81, 410.82, 410.90, |
| | | 410.91, 410.92, 411.0, 411.1, 411.81, 411.89, 412, 413.0, |
| | | 413.1, 413.9, 414.00, 414.01, 414.02, 414.03, 414.04, |
| | | 414.05, 414.06, 414.07, 414.12, 414.2, 414.3, 414.8, 414.9 |
| Substance | 660, 661 | Not applicable |
| abuse disorders | | |
| (drug and | | |
| alcohol) | | |
| Chronic | 127 | 490, 491.0, 491.1, 491.20, 491.21, 491.22, 491.8, 491.9, |
| obstructive | | 492.0, 492.8, 494.0, 494.1, 496 |
| pulmonary | | |
| disease | | |
| Osteoporosis | 206 | 733.00, 733.01, 733.02, 733.03, 733.09 |
| Chronic kidney | 158 | 016.00, 016.01, 016.02, 016.03, 016.04, 016.05, 016.06, |
| disease | | 095.4, 189.0, 189.9, 223.0, 236.91, 249.40, 249.41, 250.40, |
| | | 250.41, 250.42, 250.43, 271.4, 274.10, 283.11, 403.01, |
| | | 403.11, 403.91, 404.02, 404.03, 404.12, 404.13, 404.92, |
| | | 404.93, 440.1, 442.1, 572.4, 580.0, 580.4, 580.81, 580.89, |
| | | 580.9, 581.0, 581.1, 581.2, 581.3, 581.81, 581.89, 581.9, |
| | | 582.0, 582.1, 582.2, 582.4, 582.81, 582.89, 582.9, 583.0, |
| | | 583.1, 583.2, 583.4, 583.6, 583.7, 583.81, 583.89, 583.9, |
| | | 584.5, 584.6, 584.7, 584.8, 584.9, 585.1, 585.2, 585.3, 585.4, |
| | | 585.5, 585.6, 585.9, 586, 587, 588.0, 588.1, 588.81, 588.89, |
| | | 588.9, 591, 753.12, 753.13, 753.14, 753.15, 753.16, 753.17, |
| | | 753.19, 753.20, 753.21, 753.22, 753.23, 753.29, 794.4 |
| Stroke | 109-112 | 430, 431, 433.01, 433.11, 433.21, 433.31, 433.81, 433.91, |
| | | 434.00, 434.01,434.10, 434.11, 434.90, 434.91, 435.0, 435.1, |
| | | 435.3, 435.8, 435.9, 436, 997.02 |
| Congestive | 108 | 398.91, 402.01, 402.11, 402.91, 404.01, 404.11, 404.91, |
| heart failure | | 404.03, 404.13, 404.93, 428.0, 428.1, 428.20, 428.21, |
| | | 428.22, 428.23, 428.30, 428.31, 428.32, 428.33, 428.40, |
| | | 428.41, 428.42, 428.43, 428.9 |
| Dementia | 653 | 331.0, 331.11, 331.19, 331.2, 331.7, 290.0, 290.10, 290.11, |
| (including | | 290.12, 290.13, 290.20, 290.21, 290.3, 290.40, 290.41, |
| Alzheimer's | | 290.42, 290.43, 294.0, 294.10, 294.11, 294.8, 797 |
| and other | | |
| senile | | |
| dementias) | | |

CCC = Clinical Classification Codes; CMS = Centers for Medicare and Medicaid Services; ICD-9 = International Classification of Diseases, 9th revision; US-DHHS = US Department of Health and Human Services.

### Short-Term Outcomes (From Delivery Through 12-Weeks Postpartum)

No maternal deaths, strokes, new diagnoses of CAD, CHF or arrhythmias, or initiation of dialysis were reported. Six women with HDP were admitted to the ICU with days in ICU ranging from 2-13; one referent women was admitted to the ICU for 27 days. Four-women (all for the HDP group), developed acute kidney injury, manifested as either oliguria (i.e., urinary output of <500 mL/24 hours) or as an increase in serum creatinine by ≥0.3 mg/dL. Renal function normalized in all cases, and no woman required renal replacement therapy. Finally, women with HDP, compared to referent women, were more likely to receive a blood transfusion in the postpartum period: 38 of 57 (6.7%) versus 21 of 114 (1.8%0, respectively (p-value of <0.001).

### Statistical Analyses

Per-pregnancy HPD incidence (per 100 pregnancies) was calculated considering each pregnancy as a distinct event. Per-pregnancy incidence rates were calculated overall, by HPD subtype, and stratified by calendar year and age (<20, 20-24, 25-29, 30-34, and ≥35 years) within each subtype of HPD. The denominators used to determine the incidence, by age and calendar year, are shown in Table 4. For the per-pregnancy incidence rates, 95% confidence intervals (CI) were constructed using a Wilson score interval appropriate for a proportion estimated from clustered binary data given that women could have multiple pregnancies in the cohort. The 95% CIs for the per-woman incidence rates were constructed using an exact method for a binomial proportion. Per-woman HPD incidence (per 100 women) was calculated based on classifying each woman using the following hierarchy: Eclampsia>Preeclampsia superimposed on chronic HTN>Preeclampsia>Chronic HTN>Gestational HTN>Normotensive.

TABLE 4
Denominators used for the results by calendar year in Table 6.

| Age | | | | | | | | |
|---|---|---|---|---|---|---|---|---|
| (years) | 1976 | 1977 | 1978 | 1979 | 1980 | 1981 | 1982 | TOTAL |
| <20 | 99 | 100 | 82 | 89 | 118 | 109 | 97 | 694 |
| 20-24 | 333 | 373 | 386 | 405 | 430 | 462 | 465 | 2853 |
| 25-29 | 517 | 544 | 530 | 563 | 612 | 581 | 614 | 3961 |
| 30-34 | 227 | 214 | 250 | 275 | 276 | 303 | 320 | 1865 |
| 35+ | 56 | 52 | 61 | 74 | 79 | 82 | 85 | 489 |
| TOTAL | 1232 | 1283 | 1309 | 1406 | 1515 | 1536 | 1581 | 9862 |

Each of the 16 chronic conditions was evaluated separately, and women with the condition prior to the index date (i.e., for each matched set, the date of the exposed woman's first pregnancy complicated by HPD) were excluded from each analysis in order to evaluate de novo conditions. The duration of follow-up was calculated from the index date to the date of the condition diagnosis, last visit to a REP-affiliated provider prior to the end of the study (Dec. 31, 2017), or subsequent HPD diagnosis for the referent women. Cumulative incidence curves were estimated using the Kaplan-Meier method. Cox proportional hazards models were used to estimate hazard ratios (HRs) and corresponding 95% CIs using age as the time scale with women entering the risk set at their respective index ages. The accumulation of chronic conditions was calculated as the mean number of conditions accumulated over the follow-up after the index date and was represented graphically using Aalen-Johansen curves. Hazard ratios were computed using Anderson-Gill regression models with age as the time scale. Robust sandwich covariance estimates were used to account for either women included in both cohorts (e.g. referent women with subsequent HPD) or women with multiple pregnancies who were selected as referents more than once. Both unadjusted models and models adjusted for education, smoking, and obesity were fit. Tests of statistical significance were conducted at the two-tailed alpha level of 0.05. Statistical analyses were performed using the SAS version 9.4 software package (SAS Institute, Inc.; Cary, NC) and R software v3.4.2.

### Results

7,544 mothers were identified who had 9,862 pregnancies of ≥20 weeks' gestation between Jan. 1, 1976 and Dec. 31, 1982 while residents of Olmsted County, Minnesota for the calculation of incidence per-pregnancy (FIG. 4). During the six-year assessment period, 659 women with a total of 719 HDP pregnancies were identified. The per-pregnancy incidence rate was 7.3% (95% CI 6.8-7.9%) for HDP, including, 0.04% for eclampsia and 3.3% for preeclampsia (Table 5). The incidence per-woman calculation was based on a sub-cohort of 1,839 women for whom there was sufficient information on all of their pregnancies≥20 weeks' gestation (FIG. 4). The per-woman incidence was twice that of their incidence rates observed per-pregnancy: and 7.5% for HDP and preeclampsia, respectively (Table 5).

TABLE 5
Per-pregnancy and per-woman incidence (per 100)
of hypertensive disorders of pregnancy.

| | | Incidence | |
|---|---|---|---|
| Per-pregnancy* | N | (%) | 95% CI † |
| Any hypertensive disorder | 719 | 7.3 | 6.8-7.9 |
| of pregnancy | | | |
| Preeclampsia | 324 | 3.3 | 3.0-3.7 |
| Eclampsia | 4 | 0.04 | 0.04-0.14 |
| Preeclampsia superimposed | 22 | 0.22 | 0.17-0.38 |
| on chronic HTN | | | |
| Gestational HTN | 300 | 3.0 | 2.7-3.4 |
| Chronic HTN | 69 | 0.70 | 0.56-0.94 |
| Normotensive pregnancies | 9143 | 92.7 | 92.2-93.3 |
| Per-woman‡ | N | % | 95% CI § |
| Any hypertensive disorder | 281 | 15.3 | 14.6-17.0 |
| of pregnancy | | | |
| Preeclampsia | 138 | 7.5 | 6.3-8.8 |
| Eclampsia | 0 | 0 | 0-0 |
| Preeclampsia superimposed | 8 | 0.44 | 0.19-0.86 |
| on chronic HTN | | | |
| Gestational HTN | 110 | 6.0 | 4.9-7.2 |
| Chronic HTN | 25 | 1.4 | 0.88-2.0 |
| Normotensive | 1558 | 84.7 | 83.0-86.3 |

CI, confidence interval; HTN, hypertension
*Per-pregnancy incidence based on 9,862 pregnancies among 7,544 residents of Olmsted County who delivered during 1976-1982.
† 95% CIs for the per-pregnancy incidence rates were constructed using a Wilson score interval appropriate for a proportion estimated from clustered binary data.
‡Per-woman incidence based on 1,839 residents of Olmsted County considering all of their pregnancies.
Each woman was classified using the following hierarchy: Eclampsia > Preeclampsia superimposed on chronic HTN > Preeclampsia > Chronic HTN > Gestational HTN > Normotensive.
§ 95% CIs for the per-woman incidence rates were constructed using an exact method for a binomial proportion.

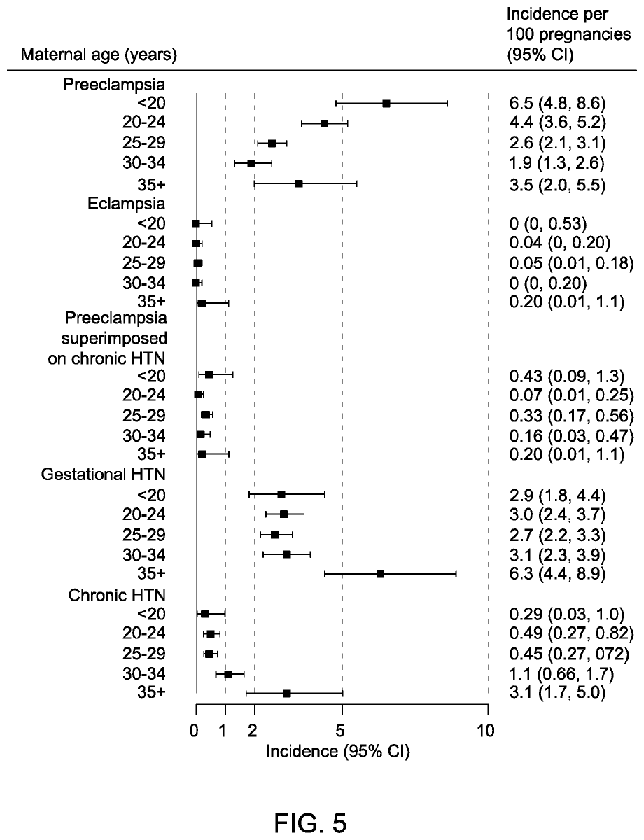

The age-specific incidence (per 100 pregnancies) of each HDP subtype is shown in FIG. 5 and in Table 6. The per-pregnancy incidence of preeclampsia with respect to age was U-shaped, such that the youngest women (i.e., <20 years and 20-24 years) and those ages≥35 years had the highest incidence. The per-pregnancy incidence of preeclampsia in women less than 20 years of age was higher than in women 20-34 years of age (6.5 [95% CI 4.8-8.6] vs 3.0 [95% CI 2.7-3.4] per 100 pregnancies). Per-pregnancy incidence of gestational hypertension was higher in women ages≥35 compared to other age groups (6.3 [95% CI 4.4-8.9] vs 2.9 [95% CI 2.5-3.2-] per 100 pregnancies.

TABLE 6
Age-specific per-pregnancy incidence (per 100 pregnancies) of hypertensive
disorders of pregnancy among 9,862 pregnancies during 1976-1982 for residents
of Olmsted County, Minnesota, overall and by calendar year.

| Hypertensive | | | | | | | | |
|---|---|---|---|---|---|---|---|---|
| disorders of | Overall | | Incidence (95% CI) by calendar year of delivery* | | | | | |
| pregnancy /Age | Incidence | | 1976-1977 | | 1978-1980 | | 1981-1982 | |
| at delivery | (95% CI)^{†} | | (N = 2,515) | | (N = 4,230) | | (N = 3,117) | |
| Preeclampsia | | | | | | | | |
| <20 | 6.5 | (4.8, 8.6) | 5.5 | (2.8, 9.7) | 7.3 | (4.6, 10.9) | 6.3 | (3.4, 10.6) |
| 20-24 | 4.4 | (3.6, 5.2) | 4.5 | (3.1, 6.3) | 4.3 | (3.2, 5.6) | 4.3 | (3.1, 5.8) |
| 25-29 | 2.6 | (2.1, 3.1) | 2.0 | (1.2, 3.0) | 2.5 | (1.8, 3.4) | 3.3 | (2.3, 4.4) |
| 30-34 | 1.9 | (1.3, 2.6) | 2.0 | (0.94, 3.8) | 1.6 | (0.9, 2.8) | 2.1 | (1.1, 3.5) |
| ≥35 | 3.5 | (2.0, 5.5) | 4.6 | (1.5, 10.5) | 3.7 | (1.6, 7.2) | 2.4 | (0.66, 6.0) |
| Eclampsia | | | | | | | | |
| <20 | 0 | (0, 0.53) | 0 | (0, 1.8) | 0 | (0, 1.3) | 0 | (0, 1.8) |
| 20-24 | 0.04 | (0, 0.20) | 0 | (0, 0.52) | 0 | (0, 0.30) | 0.11 | (0, 0.60) |
| 25-29 | 0.05 | (0.01, 0.18) | 0.09 | (0, 0.52) | 0 | (0, 0.22) | 0.08 | (0, 0.47) |
| 30-34 | 0 | (0, 0.20) | 0 | (0, 0.83) | 0 | (0, 0.46) | 0 | (0, 0.59) |
| ≥35 | 0.20 | (0.01, 1.1) | 0 | (0, 3.4) | 0.47 | (0.01, 2.6) | 0 | (0, 2.2) |
| Preeclampsia | | | | | | | | |
| superimposed on | | | | | | | | |
| chronic HTN | | | | | | | | |
| <20 | 0.43 | (0.09, 1.3) | 1.0 | (0.12, 3.6) | 0 | (0, 1.3) | 0.49 | (0.01, 2.7) |
| 20-24 | 0.07 | (0.01, 0.25) | 0 | (0, 0.52) | 0.16 | (0.02, 0.59) | 0 | (0, 0.40) |
| 25-29 | 0.33 | (0.17, 0.56) | 0.28 | (0.06, 0.82) | 0.47 | (0.2, 0.92) | 0.17 | (0.02, 0.60) |
| 30-34 | 0.16 | (0.03, 0.47) | 0.23 | (0.01, 1.3) | 0.12 | (0, 0.69) | 0.16 | (0, 0.89) |
| 35+ | 0.20 | (0.01, 1.1) | 0 | (0, 3.4) | 0 | (0, 1.7) | 0.6 | (0.02, 3.3) |
| Gestational HTN | | | | | | | | |
| <20 | 2.9 | (1.8, 4.4) | 3.5 | (1.4, 7.1) | 1.7 | (0.56, 4.0) | 3.9 | (1.7, 7.5) |
| 20-24 | 3.0 | (2.4, 3.7) | 2.4 | (1.4, 3.8) | 3.0 | (2.1, 4.1) | 3.5 | (2.4, 4.8) |
| 25-29 | 2.7 | (2.2, 3.3) | 1.8 | (1.1, 2.8) | 3.5 | (2.6, 4.4) | 2.4 | (1.6, 3.5) |
| 30-34 | 3.1 | (2.3, 3.9) | 1.1 | (0.37, 2.6) | 3.4 | (2.2, 4.9) | 4.0 | (2.6, 5.9) |
| 35+ | 6.3 | (4.4, 8.9) | 0.93 | (0.02, 5.1) | 7.9 | (4.7, 12.4) | 7.8 | (4.2, 12.9) |
| Chronic HTN | | | | | | | | |
| <20 | 0.29 | (0.03, 1.0) | 0 | (0, 1.8) | 0.35 | (0.01, 1.9) | 0.49 | (0.01, 2.7) |
| 20-24 | 0.49 | (0.27, 0.82) | 0.57 | (0.15, 1.4) | 0.57 | (0.23, 1.2) | 0.32 | (0.07, 0.94) |
| 25-29 | 0.45 | (0.27, 0.72) | 0.09 | (0, 0.52) | 0.65 | (0.32, 1.2) | 0.50 | (0.18, 1.1) |
| 30-34 | 1.1 | (0.66, 1.7) | 0.45 | (0.05, 1.6) | 1.5 | (0.78, 2.6) | 0.96 | (0.35, 2.1) |
| ≥35 | 3.1 | (1.7, 5.0) | 0.93 | (0.02, 5.1) | 3.3 | (1.3, 6.6) | 4.2 | (1.7, 8.5) |

Demographics and perinatal characteristics of HDP pregnancies are shown in Table 7. Pregnancies with preeclampsia or preeclampsia superimposed on chronic hypertension—compared to pregnancies with gestational hypertension—were less frequently carried to term, resulted more frequently in small-for-gestational age infants, and had higher frequencies of stillbirths.

TABLE 7
Demographic and perinatal characteristics of women with 719 pregnancies which met criteria for hypertensive
disorders of pregnancy during 1976-1982 for residents of Olmsted County, Minnesota.

| | Type of HDP | | | | | | | | | |
|---|---|---|---|---|---|---|---|---|---|---|
| | | | | | Preeclampsia | | | | | |
| | | | | | superimposed on | | | | | |
| | Preeclampsia | | Eclampsia | | chronic HTN | | Gestational HTN | | Chronic HTN | |
| Characteristic | (n = 324) | | (n = 4) | | (n = 22) | | (n = 300) | | (n = 69) | |
| Age at delivery (years), mean(SD) | 24.94 | (5.24) | 30.50 | (8.58) | 26.14 | (4.23) | 26.79 | (5.26) | 29.23 | (5.71) |
| BMI (kg/m²), mean(SD)* | 24.76 | (4.82) | 23.39 | (3.23) | 27.16 | (6.71) | 25.71 | (5.53) | 27.44 | (6.33) |
| Parity prior to pregnancy, n (%) † | | | | | | | | | | |
| 0 | 241 | (74.4%) | 2 | (50.0%) | 16 | (72.7%) | 151 | (50.3%) | 29 | (42.0%) |
| 1 | 50 | (15.4%) | 0 | (0.0%) | 3 | (13.6%) | 74 | (24.7%) | 17 | (24.6%) |
| 2 | 20 | (6.2%) | 0 | (0.0%) | 0 | (0.0%) | 42 | (14.0%) | 12 | (17.4%) |
| 3 or more | 13 | (4.0%) | 2 | (50.0%) | 3 | (13.6%) | 33 | (11.0%) | 11 | (15.9%) |
| History of diabetes, n (%) ‡ | 6 | (1.9%) | 0 | (0.0%) | 1 | (4.5%) | 5 | (1.7%) | 2 | (2.9%) |
| History of smoking, n (%) ‡ | 141 | (43.5%) | 0 | (0.0%) | 9 | (40.9%) | 130 | (43.3%) | 17 | (24.6%) |
| Twin pregnancies, n (%) | 13 | (4.0%) | 1 | (25.0%) | 2 | (9.1%) | 8 | (2.7%) | 2 | (2.9%) |
| Gestational weeks, n (%) | | | | | | | | | | |
| <34 weeks (pre-term) | 13 | (4.0%) | 0 | (0.0%) | 1 | (4.5%) | 1 | (0.3%) | 1 | (1.4%) |
| 34-36 weeks (pre-term) | 29 | (9.0%) | 1 | (25.0%) | 4 | (18.2%) | 13 | (4.3%) | 5 | (7.2%) |
| ≥37 weeks (term) | 282 | (87.0%) | 3 | (75.0%) | 17 | (77.3%) | 286 | (95.3%) | 63 | (91.3%) |
| Pregnancy Type, n (%) | | | | | | | | | | |
| Liveborn | 316 | (97.5%) | 4 | (100.0%) | 20 | (90.9%) | 298 | (99.3%) | 69 | (100.0%) |
| Stillborn | 8 | (2.5%) | 0 | (0.0%) | 2 | (9.1%) | 2 | (0.7%) | 0 | (0.0%) |
| Fetal weight percentile, n (%) § | | | | | | | | | | |
| ≥10% | 254/323 | (78.6%) | 0 | (0.0%) | 14/21 | (66.7%) | 265/296 | (89.5%) | 61 | (88.4%) |
| <10% | 69/323 | (21.4%) | 4 | (100.0%) | 7/21 | (33.3%) | 31/296 | (10.5%) | 8 | (11.6%) |
| APGAR 1 minute, median(IQR) § | 8.0 | (7.0, 9.0) | 7.5 | (7.0, 8.0) | 8.0 | (5.0, 8.0) | 8.0 | (7.0, 9.0) | 8.0 | (7.5, 9.0) |
| APGAR 5 minute, median(IQR) § | 9.0 | (9.0, 10.0) | 9.0 | (8.0, 9.0) | 9.0 | (8.0, 9.0) | 9.0 | (9.0, 10.0) | 9.0 | (9.0, 10.0) |
| Education, n (%) | | | | | | | | | | |
| Less than high school graduate | 31 | (9.6%) | 0 | (0.0%) | 0 | (0.0%) | 13 | (4.3%) | 2 | (2.9%) |
| High school graduate | 84 | (25.9%) | 1 | (25.0%) | 6 | (27.3%) | 83 | (27.7%) | 24 | (34.8%) |
| GED/adult diploma | 32 | (9.9%) | 1 | (25.0%) | 1 | (4.5%) | 32 | (10.7%) | 5 | (7.2%) |
| Some college | 69 | (21.3%) | 0 | (0.0%) | 7 | (31.8%) | 57 | (19.0%) | 10 | (14.5%) |
| College graduate or more | 83 | (25.6%) | 2 | (50.0%) | 6 | (27.3%) | 92 | (30.7%) | 25 | (36.2%) |
| Unknown | 25 | (7.7%) | 0 | (0.0%) | 2 | (9.1%) | 23 | (7.7%) | 3 | (4.3%) |

Abbreviations: APGAR, appearance, pulse, grimace, activity, and respiration; BMI, body mass index; GED, general equivalency diploma; HTN, hypertension.
*BMI based on weight taken closest to conception date, within 6 months prior and up to 20 gestational weeks.
† Parity defined as number of pregnancies with a gestational age >20 weeks resulting in a live or still birth.
‡ Including during current pregnancy
§ Fetal weight considered small for gestational age was defined as <10% by the Brenner 1976 growth curve
If twins, the fetal weight percentile and APGAR scores are based on the baby with the lowest birth weight.

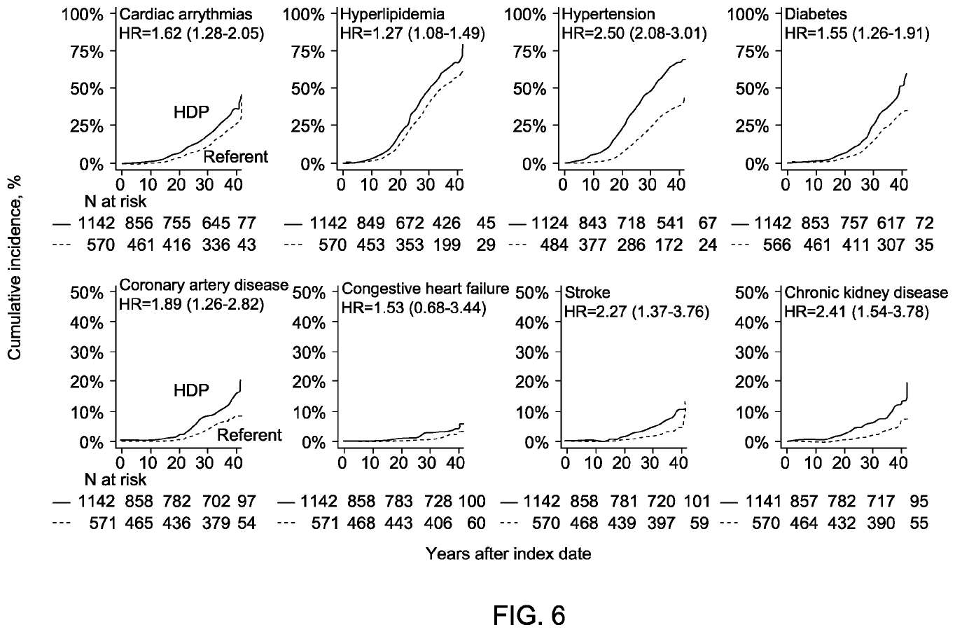

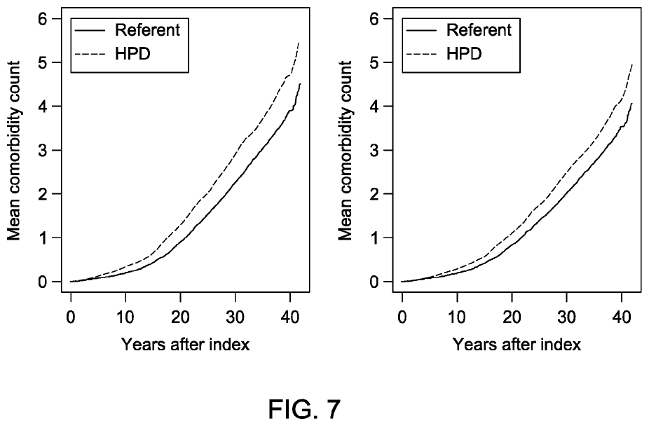

The development of chronic conditions after a pregnancy was studied in 571 women with pregnancies complicated by HDP, and 1142 age- and parity-matched referents (Table 8). The median age at last follow up was not different between the groups: 60.38 years (IQR, 52.64-64.96) for women with HDP and 59.26 (IQR, 36.79-64-47) for referent women. The median length of follow-up was 36.2 years (IQR, 23.5-38.2) and 35.8 years (IQR, 13.7-37.9) for women with a history of HDP and referent women, respectively. Women with a history of HDP, compared to referent women, demonstrated increased risks of cardiac arrhythmias (HR 1.62; 95% CI 1.28-2.05), CAD (HR 1.89; 95% CI 1.26-2.82), stroke (HR 2.27; 95% CI 1.37-3.76), hyperlipidemia (HR 1.27; 95% CI 1.08-1.49), hypertension (HR 2.50; 95% CI 2.08-3.01), diabetes (HR 1.55; 95% CI 1.26-1.91), and CKD (HR 2.41; 95% CI 1.54-3.78) in analyses adjusted for education, smoking, and obesity (Table 8 and FIG. 6). Women with a history of HDP also experienced accelerated rates of accumulation of the 16 chronic conditions considered together (Table 8 and FIG. 4). All-cause death rates were not different between the groups, but the number of deaths was small.

TABLE 8
Comparison of the incidence of chronic conditions first occurring after the index date, between
women with hypertensive disorders of pregnancy and 1:2 age- and parity-matched referent women.

| | HPD (N = 571) | | | Referent (N = 1,142) | | | | | | |
|---|---|---|---|---|---|---|---|---|---|---|
| | N at | Person- | N | N at | Person- | N | Unadjusted models | | Adjusted models* | |
| Individual Condition | risk† | years | events | risk‡ | years | events | HR (95% CI) | P | HR (95% CI) | P |
| Cardiac Arrhythmias | 570 | 15,317 | 140 | 1,142 | 28,834 | 170 | 1.63 (1.30-2.04) | <0.001 | 1.62 (1.28-2.05) | <0.001 |
| Coronary Artery Disease | 571 | 16,266 | 56 | 1,142 | 30,006 | 52 | 2.08 (1.42-3.06) | <0.001 | 1.89 (1.26-2.82) | 0.002 |
| Congestive Heart Failure | 571 | 16,726 | 16 | 1,142 | 30,382 | 14 | 2.13 (1.05-4.33) | 0.037 | 1.53 (0.68-3.44) | 0.31 |
| Stroke | 570 | 16,550 | 36 | 1,142 | 30,276 | 28 | 2.40 (1.47-3.91) | <0.001 | 2.27 (1.37-3.76) | 0.002 |
| Chronic Kidney Disease | 570 | 16,327 | 48 | 1,141 | 30,191 | 35 | 2.61 (1.69-4.04) | <0.001 | 2.41 (1.54-3.78) | <0.001 |
| Dementia | 571 | 16,741 | 13 | 1,142 | 30,390 | 16 | 1.49 (0.72-3.08) | 0.28 | 1.53 (0.72-3.23) | 0.27 |
| Depression | 569 | 13,834 | 174 | 1,139 | 25,707 | 282 | 1.14 (0.95-1.38) | 0.16 | 1.12 (0.93-1.36) | 0.24 |
| Substance abuse | 571 | 16,316 | 51 | 1,141 | 29,681 | 81 | 1.14 (0.80-1.62) | 0.47 | 1.21 (0.85-1.73) | 0.30 |
| Hyperlipidemia | 570 | 13,004 | 282 | 1,142 | 25,192 | 415 | 1.38 (1.18-1.60) | <0.001 | 1.27 (1.08-1.49) | 0.004 |
| Hypertension | 484 | 10,760 | 237 | 1,124 | 26,948 | 270 | 2.65 (2.22-3.17) | <0.001 | 2.50 (2.08-3.01) | <0.001 |
| Diabetes | 566 | 14,921 | 190 | 1,142 | 28,433 | 216 | 1.76 (1.45-2.15) | <0.001 | 1.55 (1.26-1.91) | <0.001 |
| Arthritis | 571 | 14,510 | 239 | 1,140 | 26,983 | 362 | 1.24 (1.05-1.47) | 0.011 | 1.16 (0.98-1.38) | 0.09 |
| Cancer | 568 | 15,883 | 90 | 1,139 | 28,960 | 162 | 0.99 (0.76-1.29) | 0.95 | 1.02 (0.78-1.33) | 0.90 |
| Asthma | 568 | 15,260 | 80 | 1,140 | 28,604 | 112 | 1.33 (1.00-1.78) | 0.049 | 1.25 (0.93-1.67) | 0.14 |
| COPD | 569 | 13,361 | 163 | 1,137 | 25,385 | 258 | 1.20 (0.99-1.46) | 0.071 | 1.14 (0.93-1.40) | 0.21 |
| Osteoporosis | 571 | 16,261 | 57 | 1,142 | 29,472 | 115 | 0.89 (0.65-1.22) | 0.47 | 1.01 (0.72-1.41) | 0.97 |
| Accumulation of Multimorbidity | | | | | | | | | | |
| Considering all of the above | 474 | 14,083 | — | 1,109 | 30,142 | — | 1.29 (1.20-1.39) | <0.001 | 1.25 (1.16-1.35) | <0.001 |
| Considering all of the above, | 556 | 16,541 | — | 1,125 | 30,618 | — | 1.24 (1.15-1.33) | <0.001 | 1.19 (1.11-1.29) | <0.001 |
| except hypertension | | | | | | | | | | |
| Death | | | | | | | | | | |
| All-cause | 571 | 16,913 | 33 | 1,142 | 30,561 | 45 | 1.33 (0.84-2.10) | 0.23 | 1.23 (0.78-1.96) | 0.38 |

Abbreviations: CAD, coronary artery disease; CHF, congestive heart failure; CI, confidence interval; COPD, chronic obstructive pulmonary disease; CKD, chronic kidney disease, HR, hazard ratio.
P-values in bold denote statistical significance at the 0.05 alpha level.
*Adjusted for education (categories include: <high school, high school or GED; some college; ≥college; or unknown), smoking (y/n), and obesity (defined as BMI based on weight taken closest to conception date within 6 months prior and up to 20 gestational weeks, categorized as: <25; 25-29; 30+; or unknown)
†The number of women at risk varied across conditions because women with that specific condition prior to the index date, based on the diagnosis codes considered for each condition were excluded. For hypertension, those who had hypertension prior to pregnancy based on chart review or chronic hypertension based on algorithm were also excluded. For diabetes, those who had diabetes prior to pregnancy based on chart review were also excluded. For multimorbidity, those with any of the 16 chronic conditions prior to pregnancy were excluded.

Together these results demonstrate that women with a history of HDP, compared to those with normotensive pregnancies, demonstrated significant increased risks for both kidney and heart disease, including cardiac arrhythmias, CAD, and stroke even at a relatively young median age.

### Example 3: Targeting Senescence Improves Angiogenic Potential of Adipose-Derived Mesenchymal Stem Cells in Patients with Preeclampsia

### Methods

### Participants

Women with preeclampsia and normotensive pregnant women were recruited from the Mayo Clinic Family Birth Center. All women delivered by clinically indicated Cesarean section. This study was approved by the Mayo Clinic Institutional Review Board and all participants provided written informed consent prior to participating. Each participant's medical record was reviewed to confirm the pregnancy diagnosis and obtain information on demographic characteristics and pregnancy outcomes. The diagnosis of preeclampsia was based on the presence of the following criteria: hypertension after 20 weeks gestation, defined as a blood pressure≥140/90 mmHg; b) proteinuria, defined as ≥300 mg of protein in a 24-hour urine specimen, and/or protein/creatinine (Cr) ratio of 0.3, and/or 1 +(30 mg/L) dipstick urinalysis in the absence of urinary tract infection. In the absence of proteinuria, the diagnosis of preeclampsia was confirmed if i) any of the following laboratory abnormalities were present: thrombocytopenia<100,000/μL serum Cr>1.1 mg/dL or its doubling, and elevated liver function tests, AST and ALT (>2× ULN); or ii) in the presence of pulmonary edema or cerebral or visual symptoms. Women were classified as having a normotensive pregnancy if they showed no signs of hypertension throughout gestation.

MSC isolation from adipose tissue: Abdominal fat tissue (3-5 g) obtained during C-section was cultured at 37° C./5% CO₂ in Advanced MEM media supplemented with 5% platelet lysate (PLTmax, Mill Creek Life Sciences, Rochester, MN), which provides a robust growth medium. The third passage of cells was used for phenotype/function analysis. MSC were positive to CD90, CD44, and CD105, negative to CD34, CD31, and CD45 by Flow Cytometry (FlowSight™, Amnis, Seattle, WA), and were able to transdifferentiate into adipocytes, chondrocytes, and osteocytes. For studies in non-pregnant subjects, MSC were isolated from three healthy kidney donors at time of kidney donation.

In vitro effects of TNF-alpha on MSC: MSC isolated from healthy kidney donors were treated with vehicle or 20 ng/mL TNF alpha for 24 hours. After incubation, MSC were washed and RNA isolated, and gene expression of inflammatory cytokines was measured using RT-PCR.

MSC function was assessed by proliferative and migrating capabilities. In brief, MSC migratory function was tested using a QCM™ Chemotaxis Cell Migration kit (ECM508, EMD Millipore) and proliferative activity by MTS (Promega). Proliferation and migration were measured at 490 and 560 nm, respectively, using the SynergyMX spectrophotometer (BioTek Instruments, Inc., Winooski, VT), and expressed in optical density (OD) units.

### Cell Viability

Cell viability was measured using Flow Cytometry for Annexin V.

### Immunohistochemistry

5 μm-thick sections of frozen subcutaneous adipose tissue were processed following standard protocols. Inflammation was assessed by staining for TNF-alpha (1:100, Santa Cruz Biotechnology) and monocyte chemoattractant protein (MCP)-1 (1:100, Abcam); oxidative stress was evaluated by the in situ production of superoxide anion, and detected by fluorescence microscopy using dihydroethidium (DHE). Image analysis utilized a computer-aided image-analysis program (AxioVision Carl Zeiss Micro Imaging, Thornwood, NY). Results were expressed as percent of the field-of-view staining (average of 4-6 fields).

### Apoptosis Assay for MSC

Apoptosis was assessed by Annexin V reagent (Essen Bioscience) using the IncuCyte S3 Live-Cell Analysis System (Essen Bioscience).

### Angiogenesis Assay

Angiogenic potential of MSC was assessed using human umbilical vein endothelial cells (HUVEC) angiogenesis assay. NP-MSC and PE-MSC were transferred to a 96-well plate (Corning Incorporated, USA) at 4,000 cells per well where they were co-cultured with previously seeded GFP-expressing HUVEC (IncuCyte CytoLight Green HUVEC Cells) and human fibroblasts (IncuCyte NHDF Cells) as instructed in the manufacturer's kit. The plate was placed in the IncuCyte S3 Live-Cell Analysis System where real-time images were captured every 3 hours. Angiogenesis was assessed as the total network length (mm/mm²) using IncuCyte S3 Software (Essen Bioscience) and compared between groups.

### Senescence-Associated Beta Galactosidase (SABG) Staining

For SABG staining, 50,000 MSC were seeded in a 12-well plate and left until they had reached 70-80% confluency. Cells were fixed in β-gal fixation solution for 10 minutes and washed twice with PBS. Subsequently, the cells were stained overnight using SABG reagent (Cell Signaling Technology) according to the manufacturer's instruction. Nuclei for DAPI imaging were stained using Hoechst reagent. Image analysis utilized a computer-aided image-analysis program (AxioVision Carl Zeiss Micro Imaging, Thornwood, NY). Results are shown presented as percent of the stained cells in field-of-view (average 8-10 fields).

### Analysis of Gene Expression by qPCR

MSC were collected and stored at −80° C. until further use. RNA was isolated by QIAzol Lysis Reagent and RNeasy Mini Columns (QIAGEN, Valencia, CA) following the manufacturer's instructions. RNA concentration and 230/260 absorbance ratios were checked using a Nanodrop spectrophotometer (Thermo Scientific, Wilmington, DE). cDNA was synthesized and qPCR was performed using TaqMan™ Fast Advanced Master Mix on Biorad CXF96 Platform in a 10 μL volume using the following thermal protocol: 50° C. for 2 minutes, and 45 cycles of 95° C. for 20 seconds, and 60° C. for 30 seconds. Gene expression was normalized to TATA-box-binding protein (TbP). The following primers were purchased from Applied Biosciences: total p16 (catalog number: Hs00923894), p21 (catalog number: Hs00355782), IL-6 (catalog number: Hs00174131), IL-8 (catalog number: Hs00174103), MCP-1 (catalog number: Hs00234140), PAI-1 (catalog number: Hs01126607), and PAI-2 (catalog number: Hs00299953).

### Treatment with Dasatinib

The initial dose response experiments were performed to determine the optimal Dasatinib concentration using the apoptotic assay and IncuCyte S3 Live-Cell Analysis System (Essen Bioscience). Approximately 1×10⁶ MSC (PE and NP) in passage #4, were treated with the senolytic drug, Dasatinib at concentrations of 1, 2, 5 and 10 μM (dissolved in 0.1% DMSO) for 24 hours. Three groups were analyzed: 1) cells incubated in media, 2) vehicle: cells treated with 0.1% DMSO, 3) cells treated with Dasatinib. The MSC were seeded at 5,000 cells/well in 96-well plates (Advanced MEM with 10% FBS) and treated with Dasatinib at increasing concentrations. Annexin V, added at the beginning of the treatment, labeled apoptotic cells yielding red fluorescence. The plate was scanned at a magnification of 10× and degree of fluorescence and images were assessed and taken in real time from the beginning of the treatment up to 24 hours post treatment. Using IncuCyte S3 Software a Red Object Count was generated per well at each time point. Ratios of apoptotic cells in the Dasatinib-treated, vehicle-treated, and media groups were compared and used to determine that the optimal concentration of Dasatinib is 1 μM (see Results) for studying effects of this senolytic agent on the burden of senescent PE-MSC (SABG, SASP, p16, and p21) and their functional angiogenic potential.

### Statistical Analysis

Descriptive statistics of demographic and clinical characteristics are reported as mean±SD, median and interquartile range (IQR), or number and percentage, as appropriate. Group differences between women with normotensive pregnancy and those with preeclampsia were determined by the Student t-test or and ANOVA for repeated measurements. Correlations were analyzed using Pearson correlation coefficient. Graphics for plotting individual level data were created using an interactive graph tool and GraphPad Prism 8 (RRID:SCR_002798). Correlations among various parameters were analyzed using Pearson correlation or the Spearman correlation coefficient. All data analyses were performed using SPSS statistical software, version 25 (IBM SPSS, Chicago, IL, RRID:SCR_002865), with significance determined on the basis of α=0.05.

### Results

### Clinical Characteristics of Participants

Maternal age did not differ between women with preeclampsia and those with normotensive pregnancy. Women with preeclampsia delivered earlier in pregnancy compared to women with normotensive pregnancies, and, as expected, had higher systolic and diastolic blood pressures (Table 9). Gestational diabetes and twin pregnancy, known risk factors for preeclampsia, were each documented in 20% of the PE pregnancies. Six of 10 preeclamptic pregnancies had clinical evidence of co-existing HELLP (hemolysis, elevated liver enzymes, low platelet count) syndrome

TABLE 9
Baseline characteristics of women with normotensive
versus preeclamptic pregnancies.

| | Normotensive | Preeclampsia | |
|---|---|---|---|
| Variable | (n = 12) | (n = 10) | p |
| Age at delivery, years, | 32.3 ± 5.5 | 29.5 ± 5.7 | 0.253 |
| mean ± SD | | | |
| Gestational age, weeks, | 38.6 ± 0.8 | 35.3 ± 4.0 | 0.011 |
| mean ± SD | | | |
| Systolic blood pressure, | 111.6 ± 11.5 | 152.5 ± 3.1 | <0.001 |
| mm Hg, mean ± SD | | | |
| Diastolic blood pressure, | 64.4 ± 11.0 | 100.8 ± 12.4 | <0.001 |
| mm Hg, mean ± SD | | | |
| Proteinuria, mg/24 h, | N/A | 635 (252-1917) | |
| med (IQR) | | | |
| GDM, n | | 2 | |
| Twins, n | 1 | 2 | |
| HELLP, n | | 6 | |

GDM, gestational diabetes mellitus
HELLP, hemolysis, elevated liver enzymes, low platelet count

### In Vitro Effects of TNF-Alpha on MSC From Healthy, Non Pregnant Subjects

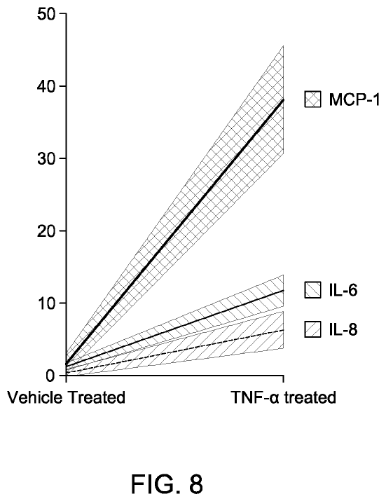

Abdominal fat tissue was obtained from three healthy kidney donors aged 39±3.3 years and a body mass index of 26.6±0.9 (mean±SEM) at time of kidney donation. MSC were isolated and characterized as described in Methods. After co-incubation with vehicle or TNF-alpha (20 ng/mL) for 24 hours, expression of the inflammatory cytokines Interleukin (IL)-6, IL-8, and MCP-1, were significantly elevated in the TNF-alpha compared to the vehicle-treated MSC (FIG. 8).

### Adipose Tissue Immunocytochemistry in Pregnancy

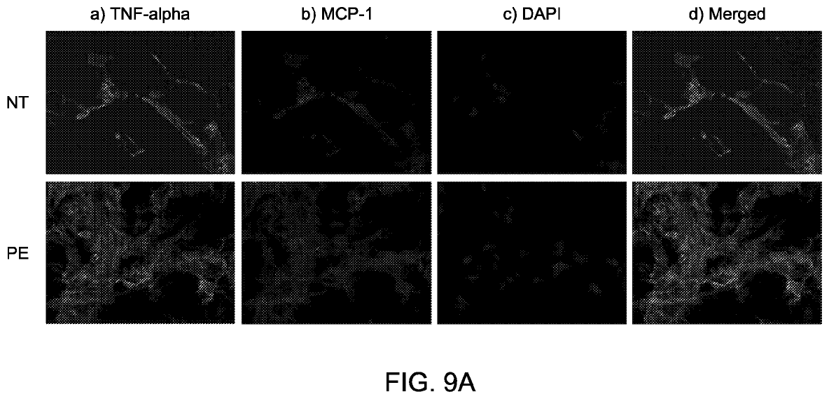

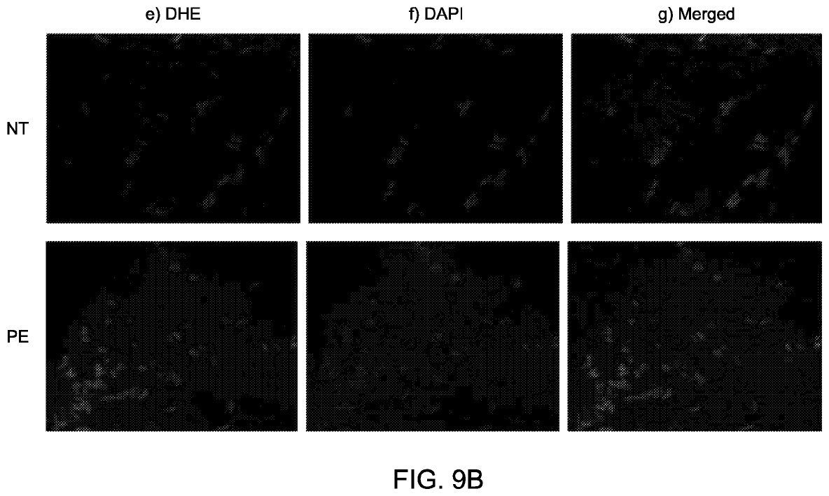

Adipose tissue staining revealed higher expression of TNF-alpha and MCP-1 in preeclampsia compared to normotensive pregnancy (Table 10, FIG. 9a, p<0.001 and p=0.024, respectively), indicating increased fat inflammation. A trend towards higher DHE staining (p=0.084) suggested a tendency for increased oxidative stress in the abdominal fat from women with preeclampsia (FIG. 9b).

TABLE 10
MSC viability, function, and immunocytochemistry of fat tissue
in women with normotensive versus preeclamptic pregnancies.

| | Normotensive/ | | | | | |
|---|---|---|---|---|---|---|
| | preeclamptic | | | | | |
| Variable | samples | Normotensive | | Preeclampsia | | p |
| Live cells, % | 12/10 | 92.47 | (91.53-93.41) | 88.50 | (85.12-91.86) | 0.012 |
| Dead cells, % | 12/10 | 4.21 | (3.57-4.86) | 7.16 | (4.41-9.91) | 0.019 |
| Proliferation (OD) * | 10/7 | 0.80 | (0.62-0.99) | 0.44 | (0.32-0.57) | 0.005 |
| Migration (OD) * | 10/7 | 1.30 | (0.99-1.61) | 1.88 | (1.42-2.33) | 0.023 |
| DHE† | 12/9 | 0.287 | (0.241-0.334) | 0.352 | (0.284-0.419) | 0.084 |
| TNF-alpha† | 12/9 | 0.090 | (0.058-0.122) | 0.284 | (0.210-0.358) | <0.001 |
| MCP-1† | 12/9 | 0.058 | (0.041-0.075) | 0.085 | (0.069-0.101) | 0.024 |

Results are expressed as mean (95% CI).
DHE, dihydroethidium; TNF-alpha, tumor necrosis factor-alpha; MCP-1, monocyte chemoattractant protein-1
* Proliferation and migration measured at 490 and 560 nm wavelengths, respectively. OD: optical density units
†Expressed as the percent of the field-of-view staining (average of 4-6 fields).

### MSC Viability, Proliferation, and Migration

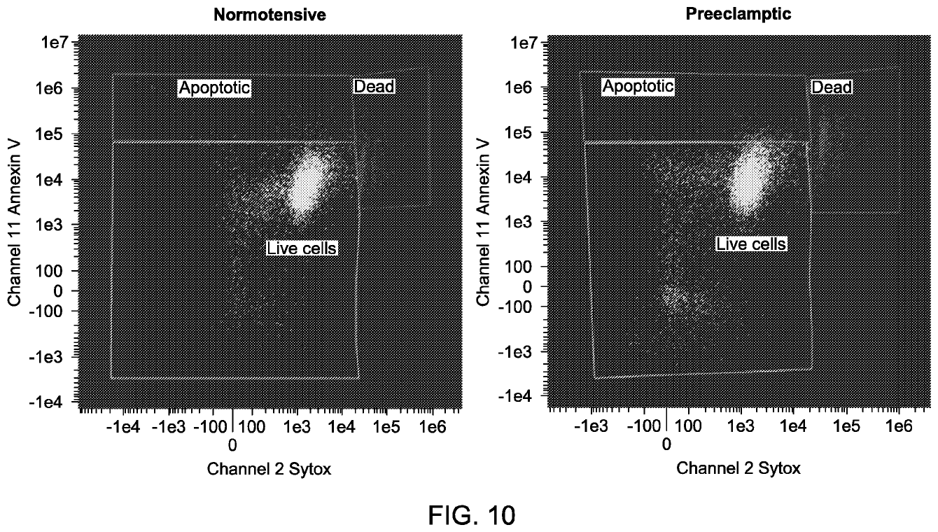

Cell viability was reduced in preeclampsia (Table 10). Women with preeclampsia had a lower percentage of live MSC cells (p=0.012) and a higher percentage of dead cells (p=0.019) than normotensive pregnant women (FIGS. 10a and 3b, Table 10). Significantly lower proliferation (p=0.005) was observed in PE-MSC compared to NP-15 MSC. In contrast, PE-MSC demonstrated higher migration (p=0.023). The average proliferation value was positively correlated with the percentage of live cells (r=0.641, p=0.006) and correlated negatively with the percentage of dead cells (r=−0.659, p=0.004).

### Higher Senescent Cell Burden, Upregulation of Senescence Markers and SASP Are Present in Preeclamptic Compared to Normotensive MSC

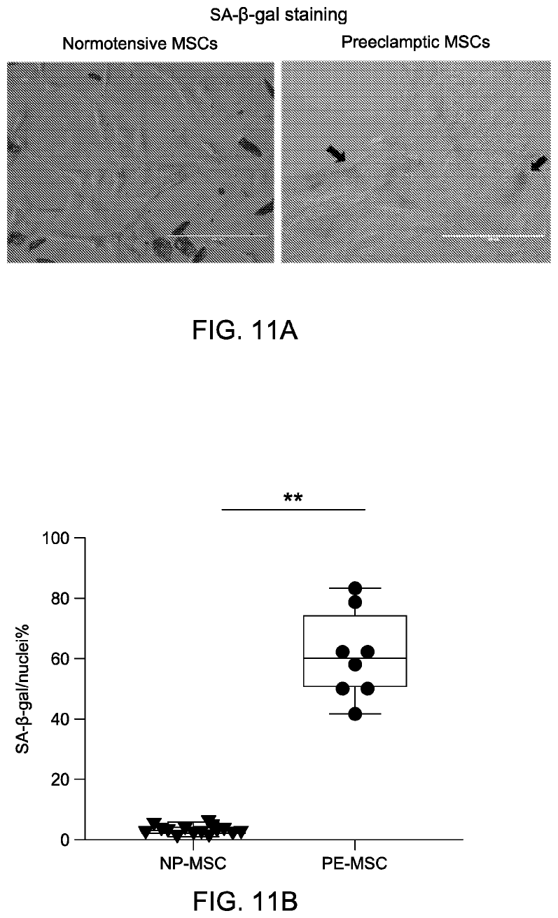

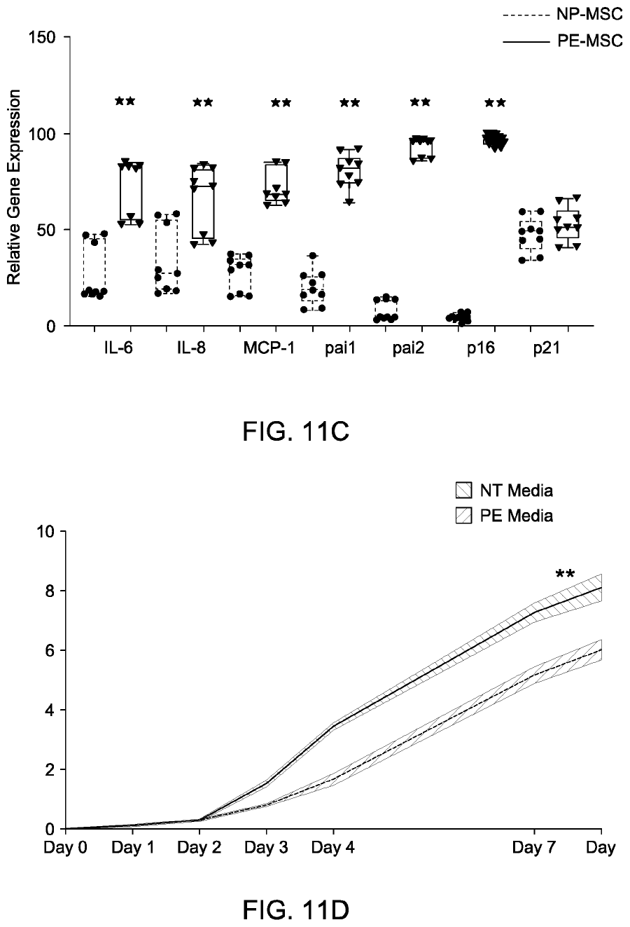

Isolated PE-MSC and NP-MSC, from preeclamptic and normotensive pregnant patients respectively, were stained for SABG. The number of senescent cells, as determined by SABG staining, was significantly higher in PE-MSC, with approximately 60.8±14.3% of counted cells being senescent, compared to 2.8±1.3% of the NP-MSC (p<0.001) (FIGS. 11a and 11b). Expression of senescence markers and SASP-related genes was assessed in both groups. PE-MSC had significantly higher expression of the senescence marker, the p16 gene (p<0.001), compared to NP-MSC, but not p21 (p=All SASP-related genes demonstrated significantly higher expression in PE-MSC compared to NP-MSC (IL-6 p<0.001, IL-8 p=0.040, MCP-1 p<0.001, PAI-1 p<PAI-2p<0.001) (FIG. 11c).

### Preeclamptic MSC Exhibit Low Angiogenic Potential

In order to examine and compare the angiogenic potential of MSC, the total network length (mm/mm²) of endothelial cells developed during co-culture with MSC was measured. PE-MSC exhibited lower angiogenic potential compared to their normotensive counterparts (p<0.001) when incubated in the medium (FIG. 11d). Monitoring the network length formation of endothelial cells was continuous for eight days, with the observation that the angiogenic potential of PE-MSC was significantly lower compared to the NP-MSC (F=13.965; df=8,p<0.001).

### Apoptotic Effects of Senolytic Agent (Dasatinib) on MSC

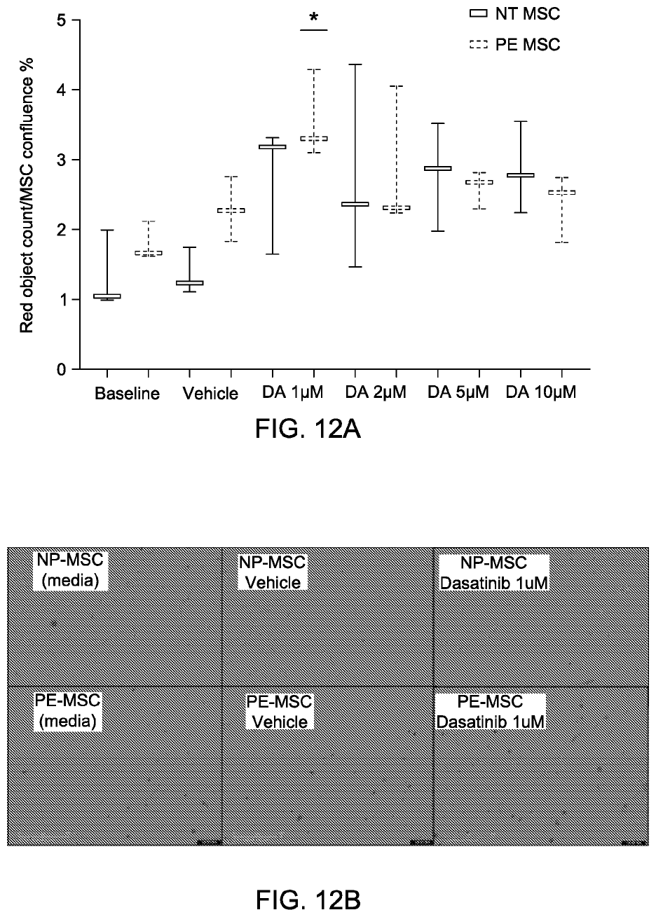

To determine the optimal senolytic drug concentration, both PE and NP-MSC were treated with four different concentrations of Dasatinib: 1 μM, 2 μM, 5μM and 10 μM. Accumulation of apoptotic bodies (Red Object Count) was assayed after 24 hours treatment in Incucyte. PE-MSC were prone to apoptosis when treated with lower concentrations of Dasatinib. Increasing concentrations of the drug did not result in a further increase in apoptosis. At the same time, NP-MSC were more sensitive to the apoptotic effects of the drug when treated with higher concentrations of Dasatinib (FIG. 12a). Notably, Dasatinib at a concentration of 1 μM induced significant apoptosis in PE-MSC (p=0.0117), but not in NP-MSC (p=0.0934), compared to the cells not treated with the drug (FIG. 12b). Based on this, 24 hours treatment with 1 μM Dasatinib was used for further experiments in this study.

### Treatment with Dasatinib Improves Angiogenic Potential of PE-MSC

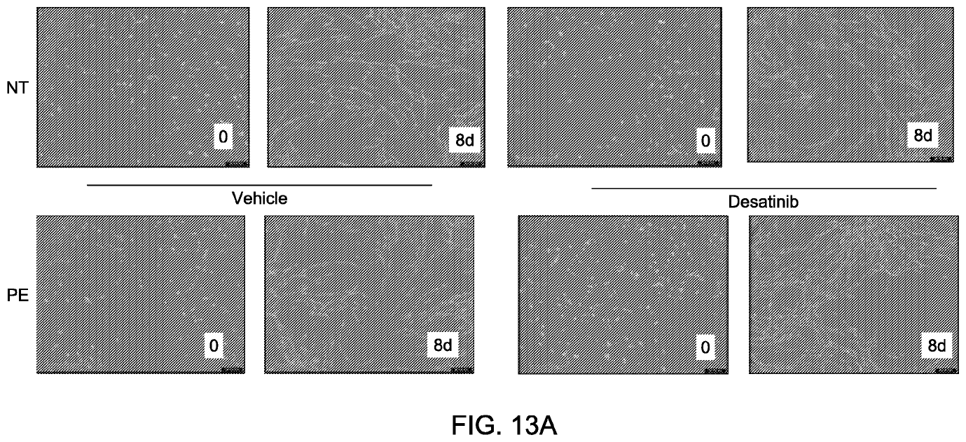

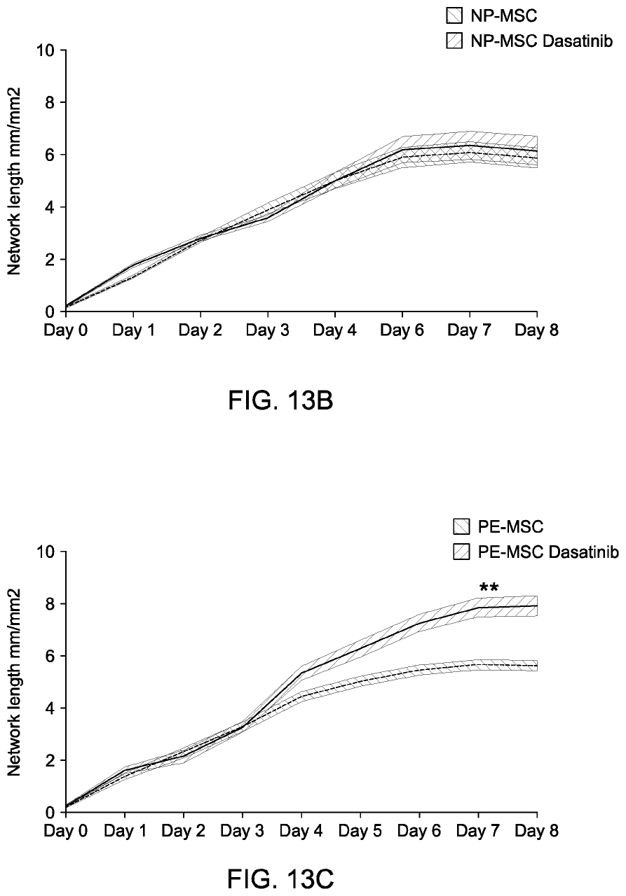

To test whether treatment with Dasatinib improves the angiogenic potential of PE-MSC, the cells were treated with this senolytic drug as described above. After the treatment, cells were co-cultured with green labelled HUVEC and total network length development was monitored for a total of 8 days (FIG. 13a). There was no significant change in the total network length of HUVECs between treated and untreated NP-MSC (FIG. 13b, F=0.406; df=8; p=0.916). During the first four days there was no significant difference in total network length of endothelial cells co-cultured with PE-MSC. However, beginning on day 5, PE-MSC treated with Dasatinib had significantly improved angiogenic potential compared to the non-treated PE mesenchymal cells (FIG. 13c, F=22.436; df=8; p<0.001).

### Treatment with Dasatinib Decreases Senescent Cell Burden and Expression of Senescence Markers and SASP Components in PE-MSC

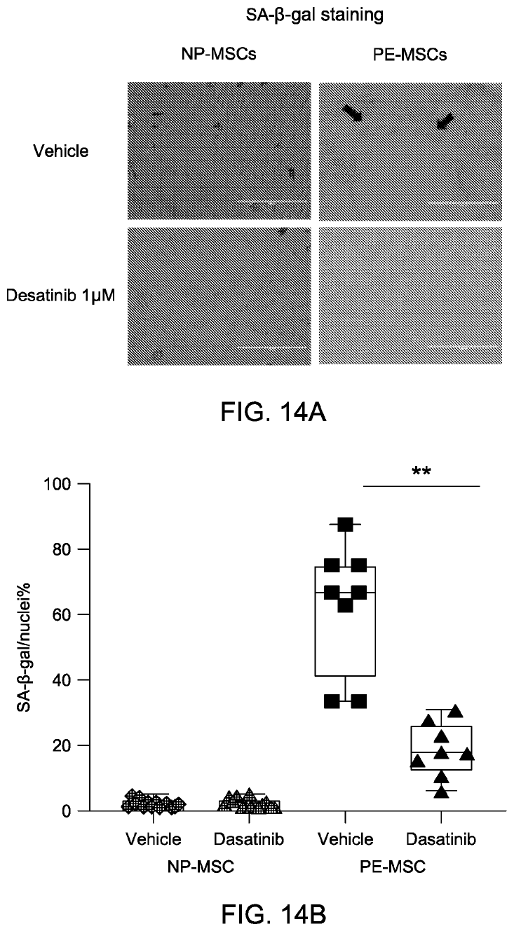

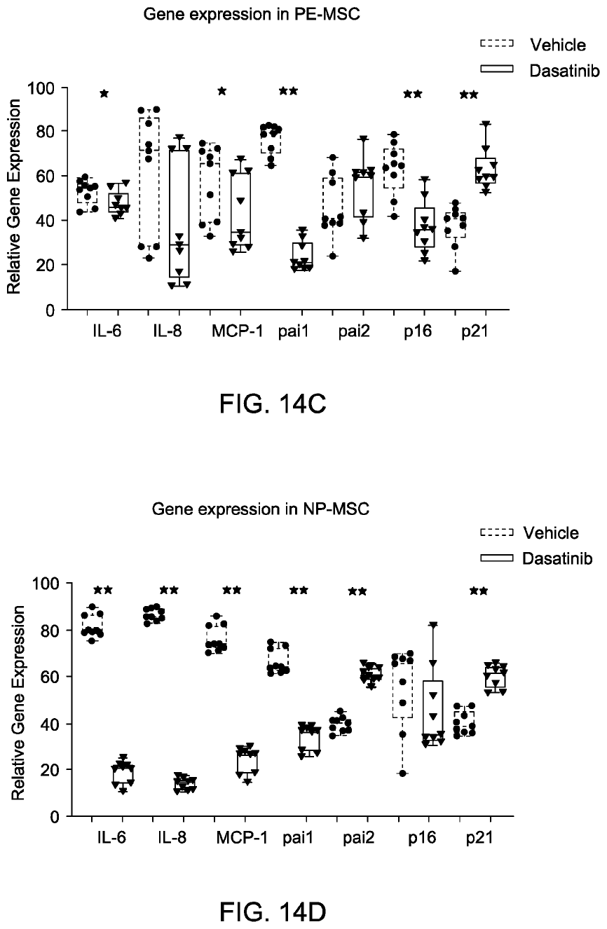

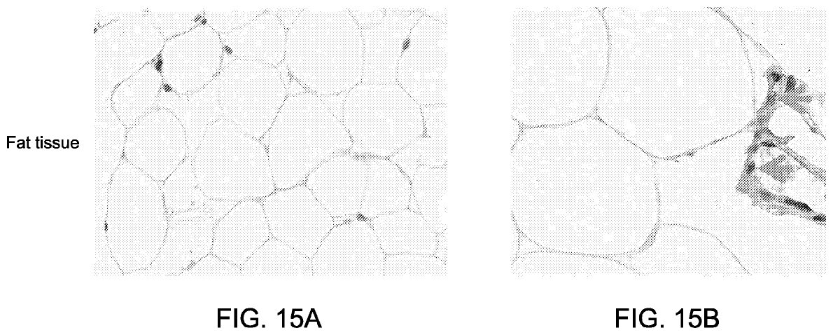

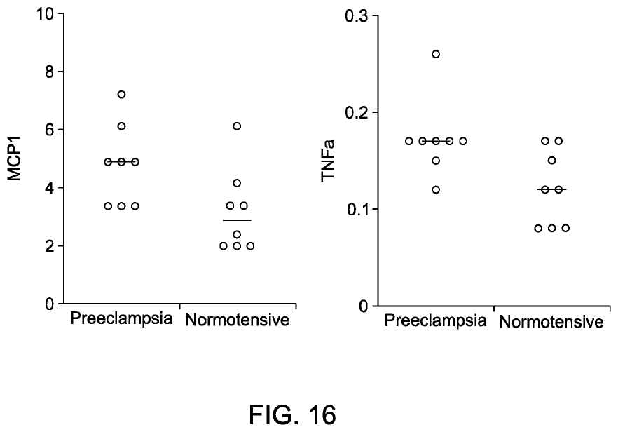

To demonstrate that Dasatinib can remove cells with the senescence phenotype, both PE-MSC and NP-MSC were stained for SABG before and after treatment with Dasatinib. The treatment with Dasatinib treatment completely removed SABG-stained cells from the culture of PE-MSC (non-treated PE-MSC=62.5±19.5% vs. treated PE-MSC=18.7±8.1%, p<0.0001) (mean±SD) (FIGS. 14a and 14b). No difference in SABG staining in NP-MSC was observed (p=0.642). In addition, Dasatinib reduced expression of senescence and SASP markers in both PE-MSC and NP-MSC. Thus, after senescent cell clearance, PE-MSC had significant decreases in expression of p16 (p<PAI-1 (p<0.001), IL-6 (p=0. 0487), and MCP-1 (p=0.040), while IL-8 (p=0.136) showed a modest decrease in expression after treatment. On the other hand, expression of p21 significantly increased (p<0.001), followed by an increase in PAI-2 gene expression (p<0.001) after treatment (FIG. 14c). Expression of the senescence marker, p16, in NP-MSC after treatment with Dasatinib remained unchanged (p=0.136). Relative gene expression of IL-6, IL-8, MCP-1, and PAI-1 was significantly decreased in NP-MSC after treatment with Dasatinib (p<0.001), while p21 and PAI2 gene expression increased (FIG. 14d).

Together these results demonstrate that a pro-inflammatory milieu of the abdominal tissue—where MSCs reside—is associated with MSC senescence, and a decrease in MSC-mediated angiogenic effects, and an increase in SASP components. These results also demonstrate that senolytics can be used to improve angiogenesis in MSCs and to decrease expression of SASP components in MSCs.

### Example 4: Preeclampsia is Associated with Upregulation of SASP Components at the Time of Delivery

13 NP and 11 PE historic serum samples collected at the time of delivery were used. SASP components were tested using the Luminex 200 system (Luminex by Eve Technologies Corp.). Women with PE pregnancies demonstrated higher levels of MCP-1, TNF-α, and plasminogen activator inhibitor (PAI)-1 (expressed in ng/mL) compared to women with NP (mean±SD: 316.95±163.95 vs. 207.53±84.78, P=.04; 1.32±0.82 vs. 0.77±0.19, P=0.03; and 58,034.73±18,854.22 vs. 38,117.15±20,856.52; P=0.02, respectively).

These results demonstrate that upregulation of SASP components continues after preeclamptic pregnancies, and can contribute to age-related comorbidities of preeclampsia such as systemic inflammation, and vascular injury and disease.

### Example 5: Role of Senescence in Preeclampsia

The gene expression level of senescence markers in the serum of normotensive and preeclamptic patients at delivery was determined as described in Example 3.

TABLE 11
Senescence markers in serum of normotensive (NP)
and preeclamptic patients (PE) at delivery.

| | Normotensive (n = 13) | Preeclamptic (n = 11) | |
|---|---|---|---|
| Marker | x ± sd | x ± sd | P value |
| NGF | 0.77 ± 0.19 | 1.30 ± 0.82 | 0.032 |
| IL6 | 15.77 ± 38.02 | 12.49 ± 16.29 | 0.793 |
| Leptin | 6106.31 ± 4509.76 | 5883.74 ± 3898.33 | 0.899 |
| IL8 | 4.81 ± 3.57 | 5.59 ± 4.23 | 0.626 |
| HGF | 512.00 ± 379.17 | 555.19 ± 264.68 | 0.754 |
| Adiponectin | 57588.85 ± 48197.79 | 65900.73 ± 48915.65 | 0.680 |
| MCP1 | 207.53 ± 84.78 | 316.95 ± 163.95 | 0.047 |
| TNFa | 2.06 ± 0.64 | 2.79 ± 1.08 | 0.053 |
| Resistin | 109215.15 ± 75908.51 | 67190.45 ± 31450.35 | 0.101 |
| Il1b | 0.34 ± 0.48 | 0.92 ± 1.50 | 0.202 |
| Pai1 | 38117.15 ± 20856.52 | 58034.73 ± 18854.22 | 0.023 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing a female mammal for a hypertensive disorder of pregnancy or the likelihood of developing said hypertensive disorder of pregnancy, wherein said method comprises:
(a) detecting the presence or absence of an elevated level of expression of a senescence-associated secretory phenotype (SASP) polypeptide in a sample from said mammal;
(b) classifying said mammal as having, or as likely to develop, said hypertensive disorder of pregnancy based at least in part on the presence of said elevated level, and
(c) classifying said mammal as lacking, or as unlikely to develop, said hypertensive disorder of pregnancy based at least in part on the absence of said elevated level.

2. The method of claim 1, wherein said mammal is a human.

3. The method of any one of claims 1-2, wherein said mammal is not pregnant.

4. The method of any one of claims 1-2, wherein said mammal is pregnant.

5. The method of any one of claims 1-4, wherein said hypertensive disorder of pregnancy is preeclampsia.

6. The method of any one of claims 1-5, wherein said SASP polypeptide is selected from the group consisting of interleukin (IL)-6, IL-8, monocyte chemotactic protein-1 (MCP-1), plasminogen activator inhibitor-1 (PAI-1), osteopontin, activine A, eutaxin, GDF15, IL1 alpha, IL1 beta, MIPa, Rantese, MMP2 miRNA, MMP3 miRNA, MMP9 miRNA, and MMP12 miRNA.

7. The method of any one of claims 1-6, wherein said sample is selected from the group consisting of whole blood, serum, plasma, peripheral blood mononucleated cells (PBMCs), urine, cerebrospinal fluid (CSF), adipose tissue, and skin tissue.

8. A method for treating a non-pregnant female mammal having a hypertensive disorder of pregnancy or likely to develop said hypertensive disorder of pregnancy, wherein said method comprises:
(a) detecting an elevated level of expression of a senescence-associated secretory phenotype (SASP) polypeptide in a sample obtained from said mammal; and
(b) administering a senolytic agent to said mammal.

9. A method for treating a hypertensive disorder of pregnancy, wherein said method comprises administering a senolytic treatment to a non-pregnant female mammal identified as having an elevated level of expression of a senescence-associated secretory phenotype (SASP) polypeptide in a sample obtained from said mammal.

10. The method of claim 8 or claim 9, wherein said mammal is a human.

11. The method of any one of claims 8-10, wherein said hypertensive disorder of pregnancy is preeclampsia.

12. The method of any one of claims 8-11, wherein said senolytic agent is dasatinib.

13. The method of any one of claims 8-12, wherein said SASP polypeptide is selected from the group consisting of interleukin (IL)-6, IL-8, monocyte chemotactic protein-1 (MCP-1), plasminogen activator inhibitor-1 (PAI-1), osteopontin, activine A, eutaxin, GDF15, IL1 alpha, IL1 beta, MIPa, Rantese, MMP2 miRNA, MMP3 miRNA, MMP9 miRNA, and MMP12 miRNA.

14. The method of any one of claims 8-13, wherein said sample is selected from the group consisting of whole blood, serum, plasma, peripheral blood mononucleated cells (PBMCs), urine, cerebrospinal fluid (CSF), adipose tissue, and skin tissue.

15. A method for delaying the onset of a comorbidity of a hypertensive disorder of pregnancy, wherein said method comprises administering a senolytic treatment to a non-pregnant female mammal (a) having had a pregnancy affected by a hypertensive disorder of pregnancy, and (b) identified as having an elevated level of expression of a senescence-associated secretory phenotype (SASP) polypeptide in a sample obtained from said mammal.

16. The method of claim 15, wherein said mammal is a human.

17. The method of any one of claims 15-16, wherein said hypertensive disorder of pregnancy is preeclampsia.

18. The method of any one of claims 15-17, wherein said senolytic agent is dasatinib.

19. The method of any one of claims 15-18, wherein said comorbidity of said hypertensive disorder of pregnancy is selected from the group consisting of diabetes mellitus type 2, cardiovascular disease, stroke, kidney disease, vascular dysfunction, atherosclerotic burden, fat inflammation, vascular injury, cardiac arrhythmias, congestive heart failure, dementia, depression, substance abuse, hyperlipidemia, arthritis, cancer, asthma, chronic obstructive pulmonary disease, and osteoporosis.

20. The method of any one of claims 15-19, wherein said SASP polypeptide is selected from the group consisting of interleukin (IL)-6, IL-8, monocyte chemotactic protein-1 (MCP-1), plasminogen activator inhibitor-1 (PAI-1), osteopontin, activine A, eutaxin, GDF15, IL1 alpha, IL1 beta, MIPa, Rantese, MMP2 miRNA, MMP3 miRNA, MMP9 miRNA, and MMP12 miRNA.

21. The method of any one of claims 15-20, wherein said sample is selected from the group consisting of whole blood, serum, plasma, peripheral blood mononucleated cells (PBMCs), urine, cerebrospinal fluid (CSF), adipose tissue, and skin tissue.
